(19) United States
(12) Patent Application Publication
(10) Pub. No.: US 2015/0377905 A1
(43) Pub. Date: Dec. 31, 2015

(54) METHODS FOR DIAGNOSIS OF KAWASAKI DISEASE

(71) Applicants: The Board of Trustees of the Leland Stanford Junior University, Palo Alto, CA (US); The Regents of the University of California, a California Corporation, Oakland, CA (US)

(72) Inventors: Jane C. Burns, La Jolla, CA (US); Harvey J. Cohen, Los Altos, CA (US); Jun Ji, Qingdao (CN); Bo Jin, Mountain View, CA (US); Bruce Xuefeng Ling, Palo Alto, CA (US); Zhou Tan, Stanford, CA (US); Adriana Tremoulet, San Diego, CA (US)

(21) Appl. No.: 14/749,604

(22) Filed: Jun. 24, 2015

Related U.S. Application Data
(60) Provisional application No. 62/016,706, filed on Jun. 25, 2014.

(51) Int. Cl. G01N 33/68

(52) U.S. Cl. CPC G01N 33/6893; G01N 2800/52; G01N 2800/328

(57) ABSTRACT

Methods for diagnosis of Kawasaki disease (KD) are disclosed. In particular, the invention relates to the use of biomarkers for aiding diagnosis, prognosis, and treatment of KD, and to a panel of biomarkers that can be used to distinguish KD from febrile illness.

### CROSS-REFERENCE TO RELATED APPLICATION

This application claims benefit under 35 U.S.C. §119(e) of provisional application 62/016,706, filed Jun. 25, 2014, which is hereby incorporated by reference in its entirety.

### TECHNICAL FIELD

The present invention pertains generally to methods for diagnosis of Kawasaki disease (KD). In particular, the invention relates to the use of biomarkers for aiding diagnosis, prognosis, and treatment of KD, and more specifically to biomarkers that can be used to distinguish KD from other febrile pediatric illnesses.

### BACKGROUND

Kawasaki Disease (KD) is the leading cause of acquired heart disease in children in the United States (Taubert et al. (1991) J. Pediatr. 119:279-282). The etiology remains unknown and there is no definitive diagnostic test. The diagnosis rests upon clinical criteria that are shared by other common pediatric illnesses (Newburger et al. (2004) Circulation 110:2747-2771). Clinical confusion can lead to a missed or delayed diagnosis, which increases the risk of coronary artery aneurysms (Wilder et al. (2007) Pediatr. Infect. Dis. J. 26:256-260; Tremoulet et al. (2008) J. Pediatr. 153:117-121). Between 15 to 30% of KD patients do not meet complete clinical criteria and are defined as having “incomplete” KD, which further contributes to delayed diagnosis (Wilder et al., supra; Tsuchiya et al. (2008) Nippon Rinsho 66:321-325; Rowley (2002) Pediatr. Infect. Dis. J. 21:563-565; Sonobe et al. (2007) Pediatr Int. 49:421-426; Anderson et al. (2005) Pediatrics 115:e428-33). Treatment with intravenous immunoglobulin (IVIG) is effective in reducing the cardiovascular complications if administered within the first 10 days after the onset of fever (Newburger et al. (1991) N. Engl. J. Med. 324:1633-1639). Without prompt treatment, approximately 25% of children with KD will develop coronary artery aneurysms, which can lead to myocardial infarction and other cardiovascular sequelae later in life (Gordon et al. (2009) J. Am. Coll. Cardiol. 54:1911-1920).

Thus, a diagnostic test for KD is urgently needed to help identify patients who require treatment and prevent cardiovascular damage.

### SUMMARY

The invention relates to the use of biomarkers for diagnosis of KD. In particular, the inventors have discovered a panel of biomarkers that can be used to diagnose KD and to distinguish KD from febrile illness. These biomarkers can be used alone or in combination with one or more additional biomarkers or relevant clinical parameters in prognosis, diagnosis, or monitoring treatment of KD.

Biomarkers that can be used in the practice of the invention include polypeptides comprising amino acid sequences from proteins including, but not limited to, lectin galactoside-binding soluble 2 (LGALS2), fucosyltransferase 7 (FUT7), matrix metallopeptidase 9 (MMP9), adrenomedullin (ADM), C-type lectin domain family 4, member D (CLEC4D), matrix metallopeptidase 8 (MMP8), natural resistance-associated macrophage protein 1 (SLC11A1), vascular endothelial growth factor A (VEGFA), and hepatocyte growth factor (HGF); and peptide fragments thereof.

In certain embodiments, a panel of biomarkers is used for diagnosis of KD. Biomarker panels of any size can be used in the practice of the invention. Biomarker panels for diagnosing KD typically comprise at least 3 biomarkers and up to 30 biomarkers, including any number of biomarkers in between, such as 3, 4, 5, 6, 7, 8, 9, 10, 11, 12, 13, 14, 15, 16, 17, 18, 19, 20, 21, 22, 23, 24, 25, 26, 27, 28, 29, or 30 biomarkers. In certain embodiments, the invention includes a biomarker panel comprising at least 3, at least 4, or at least 5, or at least 6, or at least 7, or at least 8, or at least 9, or at least 10 or more biomarkers. Although smaller biomarker panels are usually more economical, larger biomarker panels (i.e., greater than 30 biomarkers) have the advantage of providing more detailed information and can also be used in the practice of the invention.

In one embodiment, the invention includes a panel of biomarkers for diagnosing KD comprising LGALS2, FUT7, MMP9, ADM, CLEC4D, MMP8, SLC11A1, VEGFA, and HGF.

In another aspect, the invention includes a method for diagnosing KD in a patient using a biomarker panel described herein. The method comprises: a) obtaining a biological sample from the patient; b) measuring levels of each biomarker of the biomarker panel in the biological sample; and c) comparing the levels of each biomarker with respective reference value ranges for the biomarkers. The reference value ranges can represent the levels of the biomarkers for one or more samples from one or more subjects without KD (i.e., normal samples). Alternatively, the reference values can represent the levels of the biomarkers for one or more samples from one or more subjects with KD. Differential expression of the biomarkers of the biomarker panel in the biological sample compared to reference values of the biomarkers for a control subject indicate that the patient has KD. In one embodiment, the method further comprises distinguishing a diagnosis of KD from febrile illness in the patient.

In certain embodiments, clinical parameters are used for diagnosis of KD in combination with the biomarkers described herein. In one embodiment, the invention includes a method for determining a clinical score for a patient suspected of having KD. The method comprises measuring at least seven clinical parameters for the patient, including duration of fever, concentration of hemoglobin in the blood, concentration of C-reactive protein in blood, white blood cell count, percent eosinophils in the blood, percent monocytes in the blood, and percent immature neutrophils in the blood. A clinical score can be calculated using, e.g., multivariate linear discriminant analysis (LDA) from the values of the clinical parameters. The clinical score can then be classified as a low risk KD clinical score, an intermediate risk KD clinical score, or a high risk KD clinical score by methods described herein (see Example 2).

In one embodiment, the invention includes a method for diagnosing KD in a patient, the method comprising: a) determining a KD clinical score for the patient; and b) measuring the level of a plurality of biomarkers in a biological sample derived from the patient; and analyzing the levels of the biomarkers and comparing with respective reference value ranges for the biomarkers. A panel of biomarkers comprising LGALS2,FUT7, MMP9, ADM, CLEC4D, MMP8, SLC11A1, VEGFA, and HGF polypeptides, or peptide fragments thereof, may be used in combination with the clinical score for diagnosis of KD.

Methods of the invention, as described herein, can be used to distinguish a diagnosis of KD for a patient from infectious illness or acute febrile illness. A low KD clinical score indicates that a patient is unlikely to have KD, whereas a high KD clinical score indicates that a patient is highly likely to have KD. An intermediate KD clinical score for a patient can be used in combination with a biomarker expression profile for the patient to distinguish KD from infectious illness or acute febrile illness. In one embodiment, an intermediate KD clinical score is used in combination with the expression profile of a panel of biomarkers comprising LGALS2, FUT7, MMP9, ADM, CLEC4D, MMP8, SLC11A1, VEGFA, and HGF polypeptides; or peptide fragments thereof, in diagnosis of a patient.

Biomarkers can be measured, for example, by performing an enzyme-linked immunosorbent assay (ELISA), a radioimmunoassay (RIA), an immunofluorescent assay (IFA), immunohistochemistry (IHC), a sandwich assay, magnetic capture, microsphere capture, a Western Blot, surface enhanced Raman spectroscopy (SERS), flow cytometry, or mass spectrometry. In certain embodiments, the amount of a biomarker is measured by contacting an antibody with the biomarker, wherein the antibody specifically binds to the biomarker, or a fragment thereof containing an antigenic determinant of the biomarker. Antibodies that can be used in the practice of the invention include, but are not limited to, monoclonal antibodies, polyclonal antibodies, chimeric antibodies, recombinant fragments of antibodies, Fab fragments, Fab′ fragments, F(ab′)₂ fragments, Fᵥ fragments, or scFᵥ fragments.

In certain embodiments, patient data is analyzed by one or more methods including, but not limited to, multivariate linear discriminant analysis (LDA), receiver operating characteristic (ROC) analysis, ensemble data mining methods, cell specific significance analysis of microarrays (csSAM), and multi-dimensional protein identification technology (MUDPIT) analysis.

In another embodiment, the invention includes a method for evaluating the effect of an agent for treating KD in a patient using a biomarker panel described herein, the method comprising: analyzing the levels of each biomarker of the biomarker panel in samples derived from the patient before and after the patient is treated with the agent in conjunction with respective reference value ranges for each biomarker.

In another embodiment, the invention includes a method for monitoring the efficacy of a therapy for treating KD in a patient using the biomarker panel described herein, the method comprising: analyzing the levels of each biomarker of the biomarker panel in samples derived from the patient before and after the patient undergoes said therapy, in conjunction with respective reference value ranges for each biomarker.

In another embodiment, the invention includes a method of selecting a patient suspected of having KD for treatment with an intravenous immunoglobulin (IVIG), the method comprising: a) diagnosing the patient according to a method described herein, and b) selecting the patient for treatment with IVIG if the patient has a positive KD diagnosis. In one embodiment, the method comprises: a) determining the KD clinical score of the patient, and b) selecting the patient for treatment with IVIG if the patient has a KD clinical score in the high risk range or the intermediate risk range and a positive KD diagnosis based on the expression profile of a biomarker panel comprising LGALS2, FUT7, MMP9, ADM, CLEC4D, MMP8, SLC11A1, VEGFA, and HGF.

In another embodiment, the invention includes a method of treating a patient suspected of having KD, the method comprising: a) diagnosing the patient or receiving information regarding the diagnosis of the patient according to a method described herein; and b) administering a therapeutically effective amount of intravenous immunoglobulin (IVIG) to the patient if the patient has a positive KD diagnosis. In one embodiment, the method comprises: a) determining the KD clinical score of the patient; and b) administering a therapeutically effective amount of intravenous immunoglobulin (IVIG) to the subject if the subject has a high risk KD clinical score or an intermediate risk KD clinical score and a positive KD diagnosis based on the expression profile of a biomarker panel comprising LGALS2, FUT7, MMP9, ADM, CLEC4D, MMP8, SLC11A1, VEGFA, and HGF.

In another aspect, the invention includes a kit for diagnosing KD in a patient. The kit may include a container for holding a biological sample isolated from a human patient suspected of having KD, at least one agent for measuring a KD biomarker; and printed instructions for reacting the agent with the biological sample or a portion of the biological sample to measure at least one KD biomarker in the biological sample. The agents may be packaged in separate containers. The kit may further comprise one or more control reference samples and reagents for performing an immunoassay for detection of biomarkers, as described herein.

In certain embodiments, the kit comprises agents for measuring each biomarker in a biomarker panel described herein. In one embodiment, the kit comprises agents for measuring the amount of LGALS2, FUT7, MMP9, ADM, CLEC4D, MMP8, SLC11A1, VEGFA, and HGF. Furthermore, the kit may include agents for measuring biomarkers in combination with clinical parameters for diagnosis of KD.

In certain embodiments, the kit comprises reagents for performing an immunoassay. In one embodiment, the kit comprises at least one antibody selected from the group consisting of an antibody that specifically binds to LGALS2, an antibody that specifically binds to FUT7, an antibody that specifically binds to MMP9, an antibody that specifically binds to ADM, an antibody that specifically binds to CLEC4D, an antibody that specifically binds to MMP8, an antibody that specifically binds to SLC11A1, an antibody that specifically binds to VEGFA, and an antibody that specifically binds to HGF.

In another aspect, the invention includes an assay comprising: a) measuring each biomarker of a biomarker panel, described herein, in a blood, plasma, or serum sample collected from a patient suspected of having KD; and b) comparing the measured value of each biomarker of the biomarker panel in the blood, plasma, or serum sample with reference values for each biomarker for a control subject, wherein differential expression of the biomarkers in the blood, plasma, or serum sample compared to the reference values indicate that the patient has KD. In one embodiment, the assay further comprises determining a clinical score for the patient.

In one embodiment, measuring at least one biomarker comprises performing an enzyme-linked immunosorbent assay (ELISA), a radioimmunoassay (RIA), an immunofluorescent assay (IFA), immunohistochemistry (IHC), a sandwich assay, magnetic capture, microsphere capture, a Western Blot, surface enhanced Raman spectroscopy (SERS), flow cytometry, or mass spectrometry.

In certain embodiments, measuring at least one biomarker comprises contacting an antibody with the biomarker, wherein the antibody specifically binds to the biomarker, or a fragment thereof containing an antigenic determinant of the biomarker. In certain embodiments, the antibody is selected from the group consisting of a monoclonal antibody, a polyclonal antibody, a chimeric antibody, a recombinant fragment of an antibody, an Fab fragment, an Fab′ fragment, an F(ab′)₂ fragment, an Fᵥ fragment, and an scFᵥ fragment. In one embodiment, at least one antibody is selected from the group consisting of an antibody that specifically binds to LGALS2, an antibody that specifically binds to FUT7, an antibody that specifically binds to MMP9, an antibody that specifically binds to ADM, an antibody that specifically binds to CLEC4D, an antibody that specifically binds to MMP8, an antibody that specifically binds to SLC11A1, an antibody that specifically binds to VEGFA, and an antibody that specifically binds to HGF.

These and other embodiments of the subject invention will readily occur to those of skill in the art in view of the disclosure herein.

### BRIEF DESCRIPTION OF THE FIGURES

FIG. 1 shows the study design: Five Gene Expression Omnibus (GEO) vasculitis PBMC expression studies were combined for a multiplex meta-analysis to discover biomarkers. In parallel, the PubMed database with full indexed fields (release November 2012, >22 million citations) was used to identify gene markers from the entire human genome (genes: n=37,314), which strongly associated with vasculitis and KD heart lesion phenotypes to generate new hypotheses regarding KD diagnostic genes. Biomarker candidates (n=40), found both from expression meta-analysis and literature mining, were verified with available assays. A KD diagnostic classifier was developed and validated with cohort subjects (KD, n=40; febrile control (FC), n=40).

FIGS. 2A-2C show validation results for LGALS2 using ELISA assays (Mann-Whitney test p value<0.05). FIG. 2A shows a beeswarm plot of absorbance values for each KD (Case) and the FC (Control) sample. FIG. 2B shows a beeswarm plot of concentration values (pg/ml) for each KD (Case) and the FC (Control) sample. FIG. 2C shows a standard curve generated by plotting the graph using the standard concentration.

FIGS. 3A-3C show validation results for FUT7 using ELISA assays (Mann-Whitney tests p value<0.05). FIG. 3A shows a beeswarm plot of absorbance values for each KD (Case) and the FC (Control) sample. FIG. 3B shows a beeswarm plot of concentration values (pg/ml) for each KD (Case) and the FC (Control) sample. FIG. 3C shows a standard curve generated by plotting the graph using the standard concentration.

FIGS. 4A-4C show validation results for MMP9 using ELISA assays (Mann-Whitney tests p value<0.05). FIG. 4A shows a beeswarm plot of absorbance values for each KD (Case) and the FC (Control) sample. FIG. 4B shows a beeswarm plot of concentration values (pg/ml) for each KD (Case) and the FC (Control) sample. FIG. 4C shows a standard curve generated by plotting the graph using the standard concentration.

FIGS. 5A-5C show validation results for ADM using ELISA assays (Mann-Whitney tests p value<0.05). FIG. 5A shows a beeswarm plot of absorbance values for each KD (Case) and the FC (Control) sample. FIG. 5B shows a beeswarm plot of concentration values (pg/ml) for each KD (Case) and the FC (Control) sample. FIG. 5C shows a standard curve generated by plotting the graph using the standard concentration.

FIGS. 6A-6C show validation results for CLEC4D using ELISA assays (Mann-Whitney tests p value<0.05). FIG. 6A shows a beeswarm plot of absorbance values for each KD (Case) and the FC (Control) sample. FIG. 6B shows a beeswarm plot of concentration values (pg/ml) for each KD (Case) and the FC (Control) sample. FIG. 6C shows a standard curve generated by plotting the graph using the standard concentration.

FIGS. 7A-7C show validation results for MMP8 using ELISA assays (Mann-Whitney tests p value<0.05). FIG. 7A shows a beeswarm plot of absorbance values for each KD (Case) and the FC (Control) sample. FIG. 7B shows a beeswarm plot of concentration values (pg/ml) for each KD (Case) and the FC (Control) sample. FIG. 7C shows a standard curve generated by plotting the graph using the standard concentration.

FIGS. 8A-8C show validation results for SLC11A1 using ELISA assays (Mann-Whitney tests p value<0.05). FIG. 8A shows a beeswarm plot of absorbance values for each KD (Case) and the FC (Control) sample. FIG. 8B shows a beeswarm plot of concentration values (pg/ml) for each KD (Case) and the FC (Control) sample. FIG. 8C shows a standard curve generated by plotting the graph using the standard concentration.

FIGS. 9A-9C show validation results for VEGFA using ELISA assays (Mann-Whitney tests p value<0.05). FIG. 9A shows a beeswarm plot of absorbance values for each KD (Case) and the FC (Control) sample. FIG. 9B shows a beeswarm plot of concentration values (pg/ml) for each KD (Case) and the FC (Control) sample. FIG. 9C shows a standard curve generated by plotting the graph using the standard concentration.

FIGS. 10A-10C show validation results for HGF using ELISA assays (Mann-Whitney tests p value<0.05). FIG. 10A shows a beeswarm plot of absorbance values for each KD (Case) and the FC (Control) sample. FIG. 10B shows a beeswarm plot of concentration values (pg/ml) for each KD (Case) and the FC (Control) sample. FIG. 10C shows a standard curve generated by plotting the graph using the standard concentration. FIG. 11 shows a random forest model with 5-fold cross-validation

### DETAILED DESCRIPTION

The practice of the present invention will employ, unless otherwise indicated, conventional methods of pharmacology, chemistry, biochemistry, recombinant DNA techniques and immunology, within the skill of the art. Such techniques are explained fully in the literature. See, e.g., Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (3rd Edition, 2001); Methods In Enzymology (S. Colowick and N. Kaplan eds., Academic Press, Inc.).

All publications, patents and patent applications cited herein, whether supra or infra, are hereby incorporated by reference in their entireties.

I. DEFINITIONS

In describing the present invention, the following terms will be employed, and are intended to be defined as indicated below.

It must be noted that, as used in this specification and the appended claims, the singular forms “a,” “an,” and “the” include plural referents unless the content clearly dictates otherwise. Thus, for example, reference to “a biomarker” includes a mixture of two or more biomarkers, and the like.

The term “about,” particularly in reference to a given quantity, is meant to encompass deviations of plus or minus five percent.

A “biomarker” in the context of the present invention refers to a biological compound, such as a polypeptide which is differentially expressed in a sample taken from patients having KD as compared to a comparable sample taken from control subjects (e.g., a person with a negative diagnosis, normal or healthy subject). The biomarker can be a protein, a fragment of a protein, a peptide, or a polypeptide that can be detected and/or quantified. KD biomarkers include polypeptides comprising amino acid sequences from proteins including, but not limited to, lectin galactoside-binding soluble 2 (LGALS2), fucosyltransferase 7 (FUT7), matrix metallopeptidase 9 (MMP9), adrenomedullin (ADM), C-type lectin domain family 4, member D (CLEC4D), matrix metallopeptidase 8 (MMP8), natural resistance-associated macrophage protein 1 (SLC11A1), vascular endothelial growth factor A (VEGFA), and hepatocyte growth factor (HGF); and peptide fragments thereof.

The terms “polypeptide” and “protein” refer to a polymer of amino acid residues and are not limited to a minimum length. Thus, peptides, oligopeptides, dimers, multimers, and the like, are included within the definition. Both full-length proteins and fragments thereof are encompassed by the definition. The terms also include postexpression modifications of the polypeptide, for example, glycosylation, acetylation, phosphorylation, hydroxylation, oxidation, and the like.

The phrase “differentially expressed” refers to differences in the quantity and/or the frequency of a biomarker present in a sample taken from patients having, for example, KD as compared to a control subject. For example, a biomarker can be a polypeptide, which is present at an elevated level or at a decreased level in samples of patients with KD compared to samples of control subjects. Alternatively, a biomarker can be a polypeptide, which is detected at a higher frequency or at a lower frequency in samples of patients with KD compared to samples of control subjects. A biomarker can be differentially present in terms of quantity, frequency or both.

A polypeptide is differentially expressed between two samples if the amount of the polypeptide in one sample is statistically significantly different from the amount of the polypeptide in the other sample. For example, a polypeptide is differentially expressed in two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a polypeptide is differentially expressed in two sets of samples if the frequency of detecting the polypeptide in samples of patients' suffering from KD, is statistically significantly higher or lower than in control samples. For example, a polypeptide is differentially expressed in two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

The terms “subject,” “individual,” and “patient,” are used interchangeably herein and refer to any mammalian subject for whom diagnosis, prognosis, treatment, or therapy is desired, particularly humans. Other subjects may include cattle, dogs, cats, guinea pigs, rabbits, rats, mice, horses, and so on. In some cases, the methods of the invention find use in experimental animals, in veterinary application, and in the development of animal models for disease, including, but not limited to, rodents including mice, rats, and hamsters; and primates.

As used herein, a “biological sample” refers to a sample of tissue or fluid isolated from a subject, including but not limited to, for example, blood, plasma, serum, fecal matter, urine, bone marrow, bile, spinal fluid, lymph fluid, samples of the skin, external secretions of the skin, respiratory, intestinal, and genitourinary tracts, tears, saliva, milk, blood cells, organs, biopsies and also samples of in vitro cell culture constituents, including, but not limited to, conditioned media resulting from the growth of cells and tissues in culture medium, e.g., recombinant cells, and cell components.

A “test amount” of a biomarker refers to an amount of a biomarker present in a sample being tested. A test amount can be either an absolute amount (e.g., μg/ml) or a relative amount (e.g., relative intensity of signals).

A “diagnostic amount” of a biomarker refers to an amount of a biomarker in a subject's sample that is consistent with a diagnosis of KD. A diagnostic amount can be either an absolute amount (e.g., μg/ml) or a relative amount (e.g., relative intensity of signals).

A “control amount” of a biomarker can be any amount or a range of amount which is to be compared against a test amount of a biomarker. For example, a control amount of a biomarker can be the amount of a biomarker in a person without KD. A control amount can be either in absolute amount (e.g., μg/ml) or a relative amount (e.g., relative intensity of signals). The term “antibody” encompasses polyclonal and monoclonal antibody preparations, as well as preparations including hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, e.g., Huston et al. (1988) Proc Natl Acad Sci USA 85:5879-5883); dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

“Immunoassay” is an assay that uses an antibody to specifically bind an antigen (e.g., a biomarker). The immunoassay is characterized by the use of specific binding properties of a particular antibody to isolate, target, and/or quantify the antigen. An immunoassay for a biomarker may utilize one antibody or several antibodies. Immunoassay protocols may be based, for example, upon competition, direct reaction, or sandwich type assays using, for example, a labeled antibody. The labels may be, for example, fluorescent, chemiluminescent, or radioactive.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to a biomarker from specific species such as rat, mouse, or human can be selected to obtain only those polyclonal antibodies that are specifically immunoreactive with the biomarker and not with other proteins, except for polymorphic variants and alleles of the biomarker. This selection may be achieved by subtracting out antibodies that cross-react with biomarker molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane. Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

“Capture reagent” refers to a molecule or group of molecules that specifically bind to a specific target molecule or group of target molecules. For example, a capture reagent can comprise two or more antibodies each antibody having specificity for a separate target molecule. Capture reagents can be any combination of organic or inorganic chemicals, or biomolecules, and all fragments, analogs, homologs, conjugates, and derivatives thereof that can specifically bind a target molecule.

The capture reagent can comprise a single molecule that can form a complex with multiple targets, for example, a multimeric fusion protein with multiple binding sites for different targets. The capture reagent can comprise multiple molecules each having specificity for a different target, thereby resulting in multiple capture reagent-target complexes. In certain embodiments, the capture reagent is comprised of proteins, such as antibodies.

The capture reagent can be directly labeled with a detectable moiety. For example, an anti-biomarker antibody can be directly conjugated to a detectable moiety and used in the inventive methods, devices, and kits. In the alternative, detection of the capture reagent-biomarker complex can be by a secondary reagent that specifically binds to the biomarker or the capture reagent-biomarker complex. The secondary reagent can be any biomolecule, and is preferably an antibody. The secondary reagent is labeled with a detectable moiety. In some embodiments, the capture reagent or secondary reagent is coupled to biotin, and contacted with avidin or streptavidin having a detectable moiety tag.

“Detectable moieties” or “detectable labels” contemplated for use in the invention include, but are not limited to, radioisotopes, fluorescent dyes such as fluorescein, phycoerythrin, Cy-3, Cy-5, allophycoyanin, DAPI, Texas Red, rhodamine, Oregon green, Lucifer yellow, and the like, green fluorescent protein (GFP), red fluorescent protein (DsRed), cyan fluorescent protein (CFP), yellow fluorescent protein (YFP), Cerianthus orange fluorescent protein (cOFP), alkaline phosphatase (AP), beta-lactamase, chloramphenicol acetyltransferase (CAT), adenosine deaminase (ADA), aminoglycoside phosphotransferase (neo^{r}, G418^{r}) dihydrofolate reductase (DHFR), hygromycin-B-phosphotransferase (HPH), thymidine kinase (TK), lacZ (encoding α-galactosidase), and xanthine guanine phosphoribosyltransferase (XGPRT), β-glucuronidase (gus), placental alkaline phosphatase (PLAP), secreted embryonic alkaline phosphatase (SEAP), or firefly or bacterial luciferase (LUC). Enzyme tags are used with their cognate substrate. The terms also include color-coded microspheres of known fluorescent light intensities (see e.g., microspheres with xMAP technology produced by Luminex (Austin, Tex.); microspheres containing quantum dot nanocrystals, for example, containing different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), and glass microparticles with digital holographic code images (see e.g., CyVera microbeads produced by Illumina (San Diego, Calif.). As with many of the standard procedures associated with the practice of the invention, skilled artisans will be aware of additional labels that can be used.

“Diagnosis” as used herein generally includes determination as to whether a subject is likely affected by a given disease, disorder or dysfunction. The skilled artisan often makes a diagnosis on the basis of one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

“Substantially purified” refers to nucleic acid molecules or proteins that are removed from their natural environment and are isolated or separated, and are at least about 60% free, preferably about 75% free, and most preferably about 90% free, from other components with which they are naturally associated.

II. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The invention relates to the use of biomarkers either alone or in combination with clinical parameters for diagnosis of KD. In particular, the inventors have discovered a panel of biomarkers whose expression profile can be used to diagnose KD and to distinguish KD from other inflammatory diseases, including infectious illness and acute febrile illness (see Example 1). The inventors have further developed a clinical scoring system for classifying patients according to their risk of having KD based on 7 clinical parameters, including duration of fever, hemoglobin concentration, C-reactive protein concentration, white blood cell count, percent eosinophils, percent monocytes, and percent immature neutrophils (see Example 2). This clinical scoring system can be used in combination with biomarker profiles in determining appropriate treatment regimens for patients.

A. Biomarkers

Biomarkers that can be used in the practice of the invention include polypeptides comprising amino acid sequences from proteins including, but not limited to, LGALS2, FUT7, MMP9, ADM, CLEC4D, MMP8, SLC11A1, VEGFA, and HGF; and peptide fragments thereof. Differential expression of these biomarkers is associated with KD and therefore expression profiles of these biomarkers are useful for diagnosing KD and distinguishing KD from other inflammatory conditions, including infectious illness and acute febrile illness.

Accordingly, in one aspect, the invention provides a method for diagnosing KD in a subject, comprising measuring the level of a plurality of biomarkers in a biological sample derived from a subject suspected of having KD, and analyzing the levels of the biomarkers and comparing with respective reference value ranges for the biomarkers, wherein differential expression of one or more biomarkers in the biological sample compared to one or more biomarkers in a control sample indicates that the subject has KD. When analyzing the levels of biomarkers in a biological sample, the reference value ranges used for comparison can represent the levels of one or more biomarkers found in one or more samples of one or more subjects without KD (i.e., normal or control samples). Alternatively, the reference values can represent the levels of one or more biomarkers found in one or more samples of one or more subjects with KD.

The biological sample obtained from the subject to be diagnosed is typically blood, plasma, or serum, but can be any sample from bodily fluids, tissue or cells that contain the expressed biomarkers. A “control” sample, as used herein, refers to a biological sample, such as a bodily fluid, tissue, or cells that are not diseased. That is, a control sample is obtained from a normal subject (e.g. an individual known to not have KD or any condition or symptom associated with the disease). A biological sample can be obtained from a subject by conventional techniques. For example, blood can be obtained by venipuncture; urine can be spontaneously voided by a subject or collected by bladder catheterization; and solid tissue samples can be obtained by surgical techniques according to methods well known in the art.

In certain embodiments, a panel of biomarkers is used for diagnosis of KD. Biomarker panels of any size can be used in the practice of the invention. Biomarker panels for diagnosing KD typically comprise at least 3 biomarkers and up to 30 biomarkers, including any number of biomarkers in between, such as 3, 4, 5, 6, 7, 8, 9, 10, 11, 12, 13, 14, 15, 16, 17, 18, 19, 20, 21, 22, 23, 24, 25, 26, 27, 28, 29, or 30 biomarkers. In certain embodiments, the invention includes a biomarker panel comprising at least 3, at least 4, or at least 5, or at least 6, or at least 7, or at least 8, or at least 9, or at least 10 or more biomarkers. Although smaller biomarker panels are usually more economical, larger biomarker panels (i.e., greater than 30 biomarkers) have the advantage of providing more detailed information and can also be used in the practice of the invention.

In one embodiment, the invention includes a panel of biomarkers for diagnosing KD comprising LGALS2, FUT7, MMP9, ADM, CLEC4D, MMP8, SLC11A1, VEGFA, and HGF.

In certain embodiments, clinical parameters are used for diagnosis of KD in combination with the biomarkers described herein. In one embodiment, the invention includes a method for determining a clinical score for a subject suspected of having KD. The method comprises measuring at least seven clinical parameters for the subject, including duration of fever, concentration of hemoglobin in the blood, concentration of C-reactive protein in the blood, white blood cell count, percent eosinophils in the blood, percent monocytes in the blood, and percent immature neutrophils in the blood. A clinical score can be calculated using, e.g., multivariate linear discriminant analysis (LDA) from the values of the clinical parameters. The clinical score can then be classified as a low risk KD clinical score, an intermediate risk KD clinical score, or a high risk KD clinical score by methods described herein (see Example 2).

A high risk KD clinical score or a low risk KD clinical score alone is sufficient to accurately diagnose a patient as either having KD or not having KD, respectively. For patients with intermediate risk KD clinical scores, additional information is needed to diagnose the patient accurately. A sequential diagnosis method can be used, wherein the clinical score information is combined with one or more biomarker profiles to diagnose the subject. Thus, in one embodiment, the invention includes a method for diagnosing KD in a subject comprising: a) determining a KD clinical score for the subject; and b) measuring the level of a plurality of biomarkers in a biological sample derived from the subject; and analyzing the levels of the biomarkers and comparing with respective reference value ranges for the biomarkers. For example, a panel of biomarkers comprising LGALS2, FUT7, MMP9, ADM, CLEC4D, MMP8, SLC11A1, VEGFA, and HGF polypeptides or peptide fragments thereof may be used in combination with the clinical score for diagnosis of KD.

In another aspect, the invention includes an assay comprising: a) measuring each biomarker of a biomarker panel described herein in a blood, plasma, or serum sample collected from a patient suspected of having KD; and b) comparing the measured value of each biomarker of the biomarker panel in the blood, plasma, or serum with reference values for each biomarker for subjects without KD, wherein differential expression of the biomarkers in the blood, plasma, or serum compared to the reference values indicate that the patient has KD. In certain embodiments, the assay further comprises determining a clinical score, as described herein.

The methods described herein may be used to determine if a patient suspected of having KD should be treated with an intravenous immunoglobulin (IVIG). A patient is selected for treatment with IVIG if the patient has a positive KD diagnosis based on use of a biomarker panel as described herein. In one embodiment, the method comprises: a) determining the KD clinical score of the patient, and b) selecting the patient for treatment with IVIG if the patient has a KD clinical score in the high risk range or the intermediate risk range and a positive KD diagnosis based on the expression profile of a biomarker panel comprising LGALS2, FUT7, MMP9, ADM, CLEC4D, MMP8, SLC11A1, VEGFA, and HGF.

In another embodiment, the invention includes a method of treating a subject suspected of having KD, the method comprising: a) diagnosing the patient or receiving a diagnosis for the patient according to a method described herein; and b) administering a therapeutically effective amount of intravenous immunoglobulin (IVIG) to the subject if the subject has a positive KD diagnosis based on the measured values of the biomarkers present in a biological sample collected from the subject. In one embodiment, the method comprises: a) determining the KD clinical score of the patient; and b) administering a therapeutically effective amount of intravenous immunoglobulin (IVIG) to the subject if the subject has a high risk KD clinical score or an intermediate risk KD clinical score and a positive KD diagnosis based on the expression profile of a biomarker panel comprising LGALS2, FUT7, MMP9, ADM, CLEC4D, MMP8, SLC11A1, VEGFA, and HGF.

B. Detecting and Measuring Biomarkers

It is understood that the biomarkers in a sample can be measured by any suitable method known in the art. Measurement of the expression level of a biomarker can be direct or indirect. For example, the abundance levels of RNAs or proteins can be directly quantitated. Alternatively, the amount of a biomarker can be determined indirectly by measuring abundance levels of cDNAs, amplified RNAs or DNAs, or by measuring quantities or activities of RNAs, proteins, or other molecules (e.g., metabolites) that are indicative of the expression level of the biomarker. The methods for measuring biomarkers in a sample have many applications. For example, one or more biomarkers can be measured to aid in the diagnosis of KD, to determine the appropriate treatment for a subject, to monitor responses in a subject to treatment, or to identify therapeutic compounds that modulate expression of the biomarkers in vivo or in vitro.

Detecting Biomarker Proteins, Polypeptides, and Peptides

In one embodiment, the expression levels of biomarkers are determined by measuring protein, polypeptide, or peptide levels of the biomarkers. Assays based on the use of antibodies that specifically recognize the proteins, polypeptide fragments, or peptides of the biomarkers may be used for the measurement. Such assays include, but are not limited to, immunohistochemistry (IHC), western blotting, enzyme-linked immunosorbent assay (ELISA), radioimmunoassays (RIA), “sandwich” immunoassays, fluorescent immunoassays, immunoprecipitation assays, the procedures of which are well known in the art (see, e.g., Ausubel et al, eds, 1994, Current Protocols in Molecular Biology, Vol. 1, John Wiley & Sons, Inc., New York, which is incorporated by reference herein in its entirety).

Antibodies that specifically bind to a biomarker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). A biomarker antigen can be used to immunize a mammal, such as a mouse, rat, rabbit, guinea pig, monkey, or human, to produce polyclonal antibodies. If desired, a biomarker antigen can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to a biomarker antigen can be prepared using any technique which provides for the production of antibody molecules by continuous cell lines in culture. These techniques include, but are not limited to, the hybridoma technique, the human B cell hybridoma technique, and the EBV hybridoma technique (Kohler et al., Nature 256, 495-97, 1985; Kozbor et al., J. Immunol. Methods 81, 31 42, 1985; Cote et al., Proc. Natl. Acad. Sci. 80, 2026-30, 1983; Cole et al., Mol. Cell Biol. 62, 109-20, 1984).

In addition, techniques developed for the production of “chimeric antibodies,” the splicing of mouse antibody genes to human antibody genes to obtain a molecule with appropriate antigen specificity and biological activity can be used (Morrison et al., Proc. Natl. Acad. Sci. 81, 6851-55, 1984; Neuberger et al., Nature 312, 604-08, 1984; Takeda et al., Nature 314, 452-54, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions.

Alternatively, humanized antibodies can be produced using recombinant methods, as described below. Antibodies which specifically bind to a particular antigen can contain antigen binding sites which are either partially or fully humanized, as disclosed in U.S. Pat. No. 5,565,332. Human monoclonal antibodies can be prepared in vitro as described in Simmons et al., PLoS Medicine 4(5), 928-36, 2007.

Alternatively, techniques described for the production of single chain antibodies can be adapted using methods known in the art to produce single chain antibodies which specifically bind to a particular antigen. Antibodies with related specificity, but of distinct idiotypic composition, can be generated by chain shuffling from random combinatorial immunoglobin libraries (Burton, Proc. Natl. Acad. Sci. 88, 11120-23, 1991).

Single-chain antibodies also can be constructed using a DNA amplification method, such as PCR, using hybridoma cDNA as a template (Thirion et al., Eur. J. Cancer Prey. 5, 507-11, 1996). Single-chain antibodies can be mono- or bispecific, and can be bivalent or tetravalent. Construction of tetravalent, bispecific single-chain antibodies is taught, for example, in Coloma & Morrison, Nat. Biotechnol. 15, 159-63, 1997. Construction of bivalent, bispecific single-chain antibodies is taught in Mallender & Voss, J. Biol. Chem. 269, 199-206, 1994.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar et al., Int. J Cancer 61, 497-501, 1995; Nicholls et al., J. Immunol. Meth. 165, 81-91, 1993).

Antibodies which specifically bind to a biomarker antigen also can be produced by inducing in vivo production in the lymphocyte population or by screening immunoglobulin libraries or panels of highly specific binding reagents as disclosed in the literature (Orlandi et al., Proc. Natl. Acad. Sci. 86, 3833 3837, 1989; Winter et al., Nature 349, 293 299, 1991).

Chimeric antibodies can be constructed as disclosed in WO 93/03151. Binding proteins which are derived from immunoglobulins and which are multivalent and multispecific, such as the “diabodies” described in WO 94/13804, also can be prepared.

Antibodies can be purified by methods well known in the art. For example, antibodies can be affinity purified by passage over a column to which the relevant antigen is bound. The bound antibodies can then be eluted from the column using a buffer with a high salt concentration.

Antibodies may be used in diagnostic assays to detect the presence or for quantification of the biomarkers in a biological sample. Such a diagnostic assay may comprise at least two steps; (i) contacting a biological sample with the antibody, wherein the sample is a tissue (e.g., human, animal, etc.), biological fluid (e.g., blood, urine, sputum, semen, amniotic fluid, saliva, etc.), biological extract (e.g., tissue or cellular homogenate, etc.), a protein microchip (e.g., See Arenkov P, et al., Anal Biochem., 278(2):123-131 (2000)), or a chromatography column, etc; and (ii) quantifying the antibody bound to the substrate. The method may additionally involve a preliminary step of attaching the antibody, either covalently, electrostatically, or reversibly, to a solid support, before subjecting the bound antibody to the sample, as defined above and elsewhere herein.

Various diagnostic assay techniques are known in the art, such as competitive binding assays, direct or indirect sandwich assays and immunoprecipitation assays conducted in either heterogeneous or homogenous phases (Zola, Monoclonal Antibodies: A Manual of Techniques, CRC Press, Inc., (1987), pp 147-158). The antibodies used in the diagnostic assays can be labeled with a detectable moiety. The detectable moiety should be capable of producing, either directly or indirectly, a detectable signal. For example, the detectable moiety may be a radioisotope, such as ²H, ¹⁴C, ³²P, or ¹²⁵I, a fluorescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating the antibody to the detectable moiety may be employed, including those methods described by Hunter et al., Nature, 144:945 (1962); David et al., Biochem., 13:1014 (1974); Pain et al., J. Immunol. Methods, 40:219 (1981); and Nygren, J. Histochem. and Cytochem. 30:407 (1982).

Immunoassays can be used to determine the presence or absence of a biomarker in a sample as well as the quantity of a biomarker in a sample. First, a test amount of a biomarker in a sample can be detected using the immunoassay methods described above. If a biomarker is present in the sample, it will form an antibody-biomarker complex with an antibody that specifically binds the biomarker under suitable incubation conditions, as described above. The amount of an antibody-biomarker complex can be determined by comparing to a standard. A standard can be, e.g., a known compound or another protein known to be present in a sample. As noted above, the test amount of a biomarker need not be measured in absolute units, as long as the unit of measurement can be compared to a control.

It may be useful in the practice of the invention to fractionate biological samples, e.g., to enrich samples for lower abundance proteins to facilitate detection of biomarkers, or to partially purify biomarkers isolated from biological samples to generate specific antibodies to biomarkers. There are many ways to reduce the complexity of a sample based on the binding properties of the proteins in the sample, or the characteristics of the proteins in the sample.

In one embodiment, a sample can be fractionated according to the size of the proteins in a sample using size exclusion chromatography. For a biological sample wherein the amount of sample available is small, preferably a size selection spin column is used. In general, the first fraction that is eluted from the column (“fraction 1”) has the highest percentage of high molecular weight proteins; fraction 2 has a lower percentage of high molecular weight proteins; fraction 3 has even a lower percentage of high molecular weight proteins; fraction 4 has the lowest amount of large proteins; and so on. Each fraction can then be analyzed by immunoassays, gas phase ion spectrometry, and the like, for the detection of biomarkers.

In another embodiment, a sample can be fractionated by anion exchange chromatography. Anion exchange chromatography allows fractionation of the proteins in a sample roughly according to their charge characteristics. For example, a Q anion-exchange resin can be used (e.g., Q HyperD F, Biosepra), and a sample can be sequentially eluted with eluants having different pH's. Anion exchange chromatography allows separation of biomarkers in a sample that are more negatively charged from other types of biomarkers. Proteins that are eluted with an eluant having a high pH are likely to be weakly negatively charged, and proteins that are eluted with an eluant having a low pH are likely to be strongly negatively charged. Thus, in addition to reducing complexity of a sample, anion exchange chromatography separates proteins according to their binding characteristics.

In yet another embodiment, a sample can be fractionated by heparin chromatography. Heparin chromatography allows fractionation of the biomarkers in a sample also on the basis of affinity interaction with heparin and charge characteristics. Heparin, a sulfated mucopolysaccharide, will bind biomarkers with positively charged moieties, and a sample can be sequentially eluted with eluants having different pH's or salt concentrations. Biomarkers eluted with an eluant having a low pH are more likely to be weakly positively charged. Biomarkers eluted with an eluant having a high pH are more likely to be strongly positively charged. Thus, heparin chromatography also reduces the complexity of a sample and separates biomarkers according to their binding characteristics.

In yet another embodiment, a sample can be fractionated by isolating proteins that have a specific characteristic, e.g. glycosylation. For example, a CSF sample can be fractionated by passing the sample over a lectin chromatography column (which has a high affinity for sugars). Glycosylated proteins will bind to the lectin column and non-glycosylated proteins will pass through the flow through. Glycosylated proteins are then eluted from the lectin column with an eluant containing a sugar, e.g., N-acetyl-glucosamine and are available for further analysis.

In yet another embodiment, a sample can be fractionated using a sequential extraction protocol. In sequential extraction, a sample is exposed to a series of adsorbents to extract different types of biomarkers from a sample. For example, a sample is applied to a first adsorbent to extract certain proteins, and an eluant containing non-adsorbent proteins (i.e., proteins that did not bind to the first adsorbent) is collected. Then, the fraction is exposed to a second adsorbent. This further extracts various proteins from the fraction. This second fraction is then exposed to a third adsorbent, and so on. Any suitable materials and methods can be used to perform sequential extraction of a sample. For example, a series of spin columns comprising different adsorbents can be used. In another example, a multi-well comprising different adsorbents at its bottom can be used. In another example, sequential extraction can be performed on a probe adapted for use in a gas phase ion spectrometer, wherein the probe surface comprises adsorbents for binding biomarkers. In this embodiment, the sample is applied to a first adsorbent on the probe, which is subsequently washed with an eluant. Biomarkers that do not bind to the first adsorbent are removed with an eluant. The biomarkers that are in the fraction can be applied to a second adsorbent on the probe, and so forth. The advantage of performing sequential extraction on a gas phase ion spectrometer probe is that biomarkers that bind to various adsorbents at every stage of the sequential extraction protocol can be analyzed directly using a gas phase ion spectrometer.

In yet another embodiment, biomarkers in a sample can be separated by high-resolution electrophoresis, e.g., one or two-dimensional gel electrophoresis. A fraction containing a biomarker can be isolated and further analyzed by gas phase ion spectrometry. Preferably, two-dimensional gel electrophoresis is used to generate a two-dimensional array of spots for the biomarkers. See, e.g., Jungblut and Thiede, Mass Spectr. Rev. 16:145-162 (1997).

Two-dimensional gel electrophoresis can be performed using methods known in the art. See, e.g., Deutscher ed., Methods In Enzymology vol. 182. Typically, biomarkers in a sample are separated by, e.g., isoelectric focusing, during which biomarkers in a sample are separated in a pH gradient until they reach a spot where their net charge is zero (i.e., isoelectric point). This first separation step results in one-dimensional array of biomarkers. The biomarkers in the one dimensional array are further separated using a technique generally distinct from that used in the first separation step. For example, in the second dimension, biomarkers separated by isoelectric focusing are further resolved using a polyacrylamide gel by electrophoresis in the presence of sodium dodecyl sulfate (SDS-PAGE). SDS-PAGE allows further separation based on molecular mass. Typically, two-dimensional gel electrophoresis can separate chemically different biomarkers with molecular masses in the range from 1000-200,000 Da, even within complex mixtures.

Biomarkers in the two-dimensional array can be detected using any suitable methods known in the art. For example, biomarkers in a gel can be labeled or stained (e.g., Coomassie Blue or silver staining) If gel electrophoresis generates spots that correspond to the molecular weight of one or more biomarkers of the invention, the spot can be further analyzed by densitometric analysis or gas phase ion spectrometry. For example, spots can be excised from the gel and analyzed by gas phase ion spectrometry. Alternatively, the gel containing biomarkers can be transferred to an inert membrane by applying an electric field. Then a spot on the membrane that approximately corresponds to the molecular weight of a biomarker can be analyzed by gas phase ion spectrometry. In gas phase ion spectrometry, the spots can be analyzed using any suitable techniques, such as MALDI or SELDI.

Prior to gas phase ion spectrometry analysis, it may be desirable to cleave biomarkers in the spot into smaller fragments using cleaving reagents, such as proteases (e.g., trypsin). The digestion of biomarkers into small fragments provides a mass fingerprint of the biomarkers in the spot, which can be used to determine the identity of the biomarkers if desired.

In yet another embodiment, high performance liquid chromatography (HPLC) can be used to separate a mixture of biomarkers in a sample based on their different physical properties, such as polarity, charge and size. HPLC instruments typically consist of a reservoir, the mobile phase, a pump, an injector, a separation column, and a detector. Biomarkers in a sample are separated by injecting an aliquot of the sample onto the column. Different biomarkers in the mixture pass through the column at different rates due to differences in their partitioning behavior between the mobile liquid phase and the stationary phase. A fraction that corresponds to the molecular weight and/or physical properties of one or more biomarkers can be collected. The fraction can then be analyzed by gas phase ion spectrometry to detect biomarkers.

Optionally, a biomarker can be modified before analysis to improve its resolution or to determine its identity. For example, the biomarkers may be subject to proteolytic digestion before analysis. Any protease can be used. Proteases, such as trypsin, that are likely to cleave the biomarkers into a discrete number of fragments are particularly useful. The fragments that result from digestion function as a fingerprint for the biomarkers, thereby enabling their detection indirectly. This is particularly useful where there are biomarkers with similar molecular masses that might be confused for the biomarker in question. Also, proteolytic fragmentation is useful for high molecular weight biomarkers because smaller biomarkers are more easily resolved by mass spectrometry. In another example, biomarkers can be modified to improve detection resolution. For instance, neuraminidase can be used to remove terminal sialic acid residues from glycoproteins to improve binding to an anionic adsorbent and to improve detection resolution. In another example, the biomarkers can be modified by the attachment of a tag of particular molecular weight that specifically binds to molecular biomarkers, further distinguishing them. Optionally, after detecting such modified biomarkers, the identity of the biomarkers can be further determined by matching the physical and chemical characteristics of the modified biomarkers in a protein database (e.g., SwissProt).

After preparation, biomarkers in a sample are typically captured on a substrate for detection. Traditional substrates include antibody-coated 96-well plates or nitrocellulose membranes that are subsequently probed for the presence of the proteins. Alternatively, protein-binding molecules attached to microspheres, microparticles, microbeads, beads, or other particles can be used for capture and detection of biomarkers. The protein-binding molecules may be antibodies, peptides, peptoids, aptamers, small molecule ligands or other protein-binding capture agents attached to the surface of particles. Each protein-binding molecule may comprise a “unique detectable label,” which is uniquely coded such that it may be distinguished from other detectable labels attached to other protein-binding molecules to allow detection of biomarkers in multiplex assays. Examples include, but are not limited to, color-coded microspheres with known fluorescent light intensities (see e.g., microspheres with xMAP technology produced by Luminex (Austin, Tex.); microspheres containing quantum dot nanocrystals, for example, having different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), glass microparticles with digital holographic code images (see e.g., CyVera microbeads produced by Illumina (San Diego, Calif.); chemiluminescent dyes, combinations of dye compounds; and beads of detectably different sizes. See, e.g., U.S. Pat. No. 5,981,180, U.S. Pat. No. 7,445,844, U.S. Pat. No. 6,524,793, Rusling et al. (2010) Analyst 135(10): 2496-2511; Kingsmore (2006) Nat. Rev. Drug Discov. 5(4): 310-320, Proceedings Vol. 5705 Nanobiophotonics and Biomedical Applications II, Alexander N. Cartwright; Marek Osinski, Editors, pp. 114-122; Nanobiotechnology Protocols Methods in Molecular Biology, 2005, Volume 303; herein incorporated by reference in their entireties).

In another example, biochips can be used for capture and detection of proteins. Many protein biochips are described in the art. These include, for example, protein biochips produced by Packard BioScience Company (Meriden Conn.), Zyomyx (Hayward, Calif.) and Phylos (Lexington, Mass.). In general, protein biochips comprise a substrate having a surface. A capture reagent or adsorbent is attached to the surface of the substrate. Frequently, the surface comprises a plurality of addressable locations, each of which location has the capture reagent bound there. The capture reagent can be a biological molecule, such as a polypeptide or a nucleic acid, which captures other biomarkers in a specific manner. Alternatively, the capture reagent can be a chromatographic material, such as an anion exchange material or a hydrophilic material. Examples of such protein biochips are described in the following patents or patent applications: U.S. Pat. No. 6,225,047 (Hutchens and Yip, “Use of retentate chromatography to generate difference maps,” May 1, 2001), International publication WO 99/51773 (Kuimelis and Wagner, “Addressable protein arrays,” Oct. 14, 1999), International publication WO 00/04389 (Wagner et al., “Arrays of protein-capture agents and methods of use thereof,” Jul. 27, 2000), International publication WO 00/56934 (Englert et al., “Continuous porous matrix arrays,” Sep. 28, 2000).

In general, a sample containing the biomarkers is placed on the active surface of a biochip for a sufficient time to allow binding. Then, unbound molecules are washed from the surface using a suitable eluant. In general, the more stringent the eluant, the more tightly the proteins must be bound to be retained after the wash. The retained protein biomarkers now can be detected by any appropriate means, for example, mass spectrometry, fluorescence, surface plasmon resonance, ellipsometry or atomic force microscopy.

Mass spectrometry, and particularly SELDI mass spectrometry, is a particularly useful method for detection of the biomarkers of this invention. Laser desorption time-of-flight mass spectrometer can be used in embodiments of the invention. In laser desorption mass spectrometry, a substrate or a probe comprising biomarkers is introduced into an inlet system. The biomarkers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

Matrix-assisted laser desorption/ionization mass spectrometry (MALDI-MS) can also be used for detecting the biomarkers of this invention. MALDI-MS is a method of mass spectrometry that involves the use of an energy absorbing molecule, frequently called a matrix, for desorbing proteins intact from a probe surface. MALDI is described, for example, in U.S. Pat. No. 5,118,937 (Hillenkamp et al.) and U.S. Pat. No. 5,045,694 (Beavis and Chait). In MALDI-MS, the sample is typically mixed with a matrix material and placed on the surface of an inert probe. Exemplary energy absorbing molecules include cinnamic acid derivatives, sinapinic acid (“SPA”), cyano hydroxy cinnamic acid (“CHCA”) and dihydroxybenzoic acid. Other suitable energy absorbing molecules are known to those skilled in this art. The matrix dries, forming crystals that encapsulate the analyte molecules. Then the analyte molecules are detected by laser desorption/ionization mass spectrometry.

Surface-enhanced laser desorption/ionization mass spectrometry or SELDI-MS represents an improvement over MALDI for the fractionation and detection of biomolecules, such as proteins, in complex mixtures. SELDI is a method of mass spectrometry in which biomolecules, such as proteins, are captured on the surface of a protein biochip using capture reagents that are bound there. Typically, non-bound molecules are washed from the probe surface before interrogation. SELDI is described, for example, in: U.S. Pat. No. 5,719,060 (“Method and Apparatus for Desorption and Ionization of Analytes,” Hutchens and Yip, Feb. 17, 1998,) U.S. Pat. No. 6,225,047 (“Use of Retentate Chromatography to Generate Difference Maps,” Hutchens and Yip, May 1, 2001) and Weinberger et al., “Time-of-flight mass spectrometry,” in Encyclopedia of Analytical Chemistry, R. A. Meyers, ed., pp 11915-11918 John Wiley & Sons Chichesher, 2000.

Biomarkers on the substrate surface can be desorbed and ionized using gas phase ion spectrometry. Any suitable gas phase ion spectrometer can be used as long as it allows biomarkers on the substrate to be resolved. Preferably, gas phase ion spectrometers allow quantitation of biomarkers. In one embodiment, a gas phase ion spectrometer is a mass spectrometer. In a typical mass spectrometer, a substrate or a probe comprising biomarkers on its surface is introduced into an inlet system of the mass spectrometer. The biomarkers are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of biomarkers or other substances will typically involve detection of signal intensity. This, in turn, can reflect the quantity and character of biomarkers bound to the substrate. Any of the components of a mass spectrometer (e.g., a desorption source, a mass analyzer, a detector, etc.) can be combined with other suitable components described herein or others known in the art in embodiments of the invention.

Measuring Clinical Blood Parameters

Automated hematology analyzers are commonly used in clinical laboratories for determining complete blood counts (e.g., red blood cell count, white blood cell count, platelet count, and absolute neutrophil count) and erythrocyte sedimentation rates. Hematology analyzers typically count cells using cell flow cytometry techniques relying on electrical impedance, light scattering, or fluorescence to differentiate cell types.

The number of cells in a biological sample can be determined by any suitable method known in the art, including visual counting of cells observed microscopically or automated methods of cell counting. For example, cells can be counted by using a flow cytometer, Coulter counter, CASY counter, hemocytometer, or microscopic imaging. Cells can be distinguished by their shape, intracellular structures, staining characteristics, and the presence of cell markers. In particular, cell markers can be detected using methods, including but not limited to immunofluorescent antibody assay (IFA), enzyme-linked immuno-culture assay (ELICA), flow cytometry, cytometry by time-of-flight (CyTOF), and magnetic cell sorting. See. e.g., Stewart et al. (2000) Methods Cell Sci. 22(1):67-78; Cunningham (2010) Methods Mol. Biol. 588:319-339; herein incorporated by reference.

For example, various visual counting methods can be used. A hemocytometer can be used to count cells viewed under a microscope. The hemocytometer contains a grid to allow manual counting of the number of cells in a certain area and a determination of the concentration of cells in a sample. Alternatively, cells can be plated on a petri dish containing a growth medium. The cells are plated at a dilution such that each cell gives rise to a single colony. The colonies can then be visually counted to determine the concentration of particular cells types that were present in a sample.

Automated cell counting can be performed with a flow cytometer, Coulter counter, CASY counter, or by automated microscopic imaging analysis. Coulter and CASY counters can be used to measure the volumes and numbers of cells. Flow cytometry can be used for automated cell counting and sorting and for detecting surface and intracellular markers. Additionally, microscopic analysis of cells can be automated. For example, microscopy images can be analyzed using statistical classification algorithms that automate cell detection and counting. See, e.g., Shapiro (2004) Cytometry A 58(1):13-20; Glory et al. (2007) Cell Mol. Biol. 53(2):44-50; Han et al. (2012) Machine Vision and Applications 23 (1): 15-24; herein incorporated by reference.

In particular, flow cytometry can be used to distinguish subpopulations of cells expressing different cellular markers and to determine their frequency in a population of cells. Typically, whole cells are incubated with antibodies that specifically bind to the cellular markers. The antibodies can be labeled, for example, with a fluorophore, isotope, or quantum dot to facilitate detection of the cellular markers. The cells are then suspended in a stream of fluid and passed through an electronic detection apparatus. In addition, fluorescence-activated cell sorting (FACS) can be used to sort a heterogeneous mixture of cells into separate containers. (See, e.g., Shapiro Practical Flow Cytometry, Wiley-Liss, 4^{th} edition, 2003; Loken Immunofluorescence Techniques in Flow Cytometry and Sorting, Wiley, 2^{nd} edition, 1990; Flow Cytometry: Principles and Applications, (ed. Macey), Humana Press 1^{st} edition, 2007; herein incorporated by reference in their entireties.)

Cytometry by time-of-flight (CyTOF), also known as mass cytometry, is another method that can be used for detection of cellular markers in whole cells. CyTOF uses transition element isotopes as labels for antibodies, which are detected by a time-of-flight mass spectrometer. Unlike conventional flow cytometry, CyTOF is destructive to cells, but has the advantage that it can be used to analyze more cell markers simultaneously. See, e.g., Bendall et al. (2012) Trends in Immunology 33:323-332; Newell et al. (2012) Immunity 36(1):142-52; Ornatsky et al. (2010) J. Immunol. Methods 361 (1-2):1-20; Bandura et al. (2009) Analytical Chemistry 81:6813-6822; Chen et al. (2012) Cell Mol. Immunol. 9(4):322-323; and Cheung et al. (2011) Nat. Rev. Rheumatol. 7(9):502-3; herein incorporated by reference in their entireties.

The erythrocyte sedimentation rate can be determined by standard methods well-known in the art. The erythrocyte sedimentation rate is measured by collecting anticoagulated blood from a subject and determining the rate at which red blood cells fall to the bottom of a tube (e.g., Westergren tube). The sedimentation rate is commonly determined using an automated analyzer. See, e.g., International Council for Standardization in Haematology (Expert Panel on Blood Rheology) (1993) ICSH recommendations for measurement of erythrocyte sedimentation rate, International Council for Standardization in Haematology (Expert Panel on Blood Rheology) J. Clin. Pathol. 46 (3):198-203; Westergren (1957) Triangle 3 (1):20-25; Bottiger et al. (1967) Br. Med. J. 2 (5544):85-87; Miller et al. (1983) Br. Med. J. (Clin. Res. Ed.) 286 (6361): 266; herein incorporated by reference in their entireties.

C. Kits

In yet another aspect, the invention provides kits for diagnosing KD, wherein the kits can be used to measure the biomarkers of the present invention. For example, the kits can be used to detect or measure any one or more of the biomarkers described herein that distinguish a patient with KD from normal subjects. The kit may include one or more agents for measuring biomarkers, a container for holding a biological sample isolated from a human subject suspected of having KD; and printed instructions for reacting agents with the biological sample or a portion of the biological sample to measure at least one KD biomarker in the biological sample. The agents may be packaged in separate containers. The kit may further comprise one or more control reference samples and reagents for performing an immunoassay.

In certain embodiments, the kit comprises agents for measuring each biomarker in a biomarker panel described herein. In one embodiment, the kit comprises agents for measuring the amounts of LGALS2, FUT7, MMP9, ADM, CLEC4D, MMP8, SLC11A1, VEGFA, and HGF. Furthermore, the kit may include agents for measuring the biomarkers in combination with clinical parameters for diagnosis of KD.

In certain embodiments, the kit comprises reagents for performing an immunoassay. In one embodiment, the kit comprises at least one antibody selected from the group consisting of an antibody that specifically binds to LGALS2, an antibody that specifically binds to FUT7, an antibody that specifically binds to MMP9, an antibody that specifically binds to ADM, an antibody that specifically binds to CLEC4D, an antibody that specifically binds to MMP8, an antibody that specifically binds to SLC11A1, an antibody that specifically binds to VEGFA, and an antibody that specifically binds to HGF.

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of diagnosing KD.

The kits of the invention have a number of applications. For example, the kits can be used to determine if a subject has KD or some other inflammatory condition arising, for example, from an infectious illness or acute febrile illness. In another example, the kits can be used to determine if a patient should be treated with IVIG. In another example, kits can be used to monitor the effectiveness of treatment of a patient having KD. In a further example, the kits can be used to identify compounds that modulate expression of one or more of the biomarkers in in vitro or in vivo animal models to determine the effects of treatment.

III. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Novel Data-Mining Approach Identifies Biomarkers for Diagnosis of Kawasaki Disease

Introduction

Differing histopathologically from other rash or febrile illnesses caused by viral or bacterial infection and other inflammatory diseases, KD is the most commonly encountered pediatric vasculitis syndrome in the medium-sized muscular arteries (Fujiwara et al. (1978) Pediatrics 61(1):100-107; Hirose et al. (1978) European Journal of Pediatrics 129(1):17-27). Peripheral blood mononuclear cell (PBMC) gene expression is altered in both KD and other types of vasculitis patients (Kobayashi et al. (2008) Japanese Journal of Clinical Medicine 66(2):332-337). Considerable vasculitis microarray data, including from KD, have been deposited into international repositories, e.g. GEO and ArrayExpress (Popper et al. (2007) Genome Biology 8(12):R261; Popper et al. (2009) The Journal of Infectious Diseases 200(4):657-666; Kobayashi et al., supra). The characteristic necrotizing vasculitis, associated with KD suggests specific patterns of blood proteins (biomarkers) may be associated with the disease. Therefore, we hypothesized that proteins in serum participating in the vascular pathology might provide better diagnostic utility to differentiate KD from febrile control (FC) subjects.

PubMed Central is a rich resource for mining various facts of biomedicine and nature. Genes, at a genome scale, can be correlated with targeted disease phenotypes, and keywords can be used to generate new hypotheses and identify genes and gene networks, which may underlie the disease phenotypes to derive candidates for drug targets or disease diagnostic biomarkers. High throughput literature-mining methods can enable researchers to identify biological entities, e.g. gene and symptom, that co-occur within publications using a frequency-based scoring scheme to rank the extracted relationships (Jensen et al. (2006) Nature Reviews Genetics 7(2):119-129).

In this study, we tested the hypothesis that meta-analysis of the GEO vasculitis expression data and literature association study could uncover candidate biomarkers significantly associated with KD and other vasculitis diseases. These candidates were later verified with KD and FC serum samples to gauge whether they could be of potential diagnostic utility. We further hypothesized that integration of the novel KD serum biomarkers with our previously developed clinical score could effect an improved KD diagnostic algorithm (Ling et al. (2013) The Journal of Pediatrics 162(1):183-188 e183; Ling et al. (2011) BMC Med. 9:130; herein incorporated by reference in their entireties).

Materials and Methods

Patient Demographics and Samples

Informed consent was obtained from the parents of all subjects and assent from all subjects>6 years of age. This study was approved by the human subjects protection programs at the University of California San Diego and Stanford University. Inclusion criteria for KD subjects were based on the American Heart Association Guidelines (Newburger et al. (2004) Pediatrics 114(6):1708-1733). All KD subjects had fever for at least 3 days and 4 of 5 classic criteria or 3 or fewer criteria with coronary artery abnormalities documented by echocardiogram. FC subjects were age-similar children evaluated for fever accompanied by at least one of the KD criteria (rash, conjunctival injection, oral mucosa changes, extremity changes, enlarged cervical lymph node). Febrile children with prominent respiratory or gastrointestinal symptoms were specifically excluded such that the majority of the controls had KD in the differential diagnosis of their condition. All subjects provided samples of blood and urine and underwent other diagnostic tests at the discretion of the managing clinicians. De-identified clinical laboratory test data were extracted from the UCSD KD electronic database for multivariate analysis. FC patients had a clinically or culture proven etiology for their febrile illnesses or underwent resolution of fever and clinical signs within 3 days of obtaining their clinical samples (designated as “viral syndrome”).

Multiplex Meta-Analysis of Vasculitis PBMC Expression Datasets

Considerable vasculitis microarray data (KD: GSE18606, GSE9863, GSE9864 cohort 1, GSE9864 cohort 2; Takayasu's vasculitis (TA): GSE33910, GSE16945; Behcet's disease (BC): GSE17114; Popper et al. (2007), supra; Popper et al. (2009), supra; Kobayashi et al., supra; herein incorporated by reference) were combined and subjected to multiplex meta-analysis with the method we previously developed (Chen et al. (2010) PLoS Computational Biology 6(9); Morgan et al. (2010) BMC Bioinformatics 11 Suppl 9:S6; herein incorporated by reference). For each of the genes tested, we calculated the meta-fold across all studies. Significant genes were selected if they were measured with a meta-effect p value less than 4.5×10⁻⁵. We then filtered the gene sets through a list of 3,638 proteins with known detectable abundance in serum, plasma, or urine (Dudley et al. (2009) Pacific Symposium on Biocomputing 2009:27-38).

Literature Mining for Information Retrieval (IR) and Entity Recognition (ER)

Human HUGO gene names (n=37,314) were extracted with biomaRt library (BioMart project, biomart.org, version 0.8). Co-occurrence between entire 37,314 human genome gene symbols and the literature indexed keywords (“Kawasaki disease”, “Aneurysms”, “Coronary artery lesions”, “Myocardial infarction”, and “vasculitis”) in PubMed database full indexed fields (release November 2012, >22 million citations) was computed as previously described (Jensen et al. (2006) Nature Reviews. Genetics 7(2):119-129; Korbel et al. (2005) PLoS Biology 3(5):e134; herein incorporated by reference). To develop hypotheses of genes that could strongly associate with a specific outcome phenotype, we considered the ranking of the top 0.5 percent of genes for each gene-keyword co-occurrence as having significant gene-phenotype associations.

ELISA Assays Validating KD Marker Candidates

All assays were ELISA assays, and performed using commercial kits following vendor instructions. All assays were performed to measure serum levels of selected analytes: ABCC1, ADM, ALB, C19orf59, CIS, CAMK4, CD274, CD55, CD59, CLEC4D, CR1, CRTAM, CTGF, FCGR1B, FKBP1A, FKBP5, FKBP6, FUT7, HGF, HP, IFI30, LCN2, LGALS2, LILRA5, MAPK14, MMP8, MPO, MYD88, NKTR, Notch4, PCOLCE2, PPARG, PVRL2, S100Al2, S100A8, S100A9, SLC11A, TLR7, TREML4, and VEGFA.

Statistical Analyses

Patient demographic data was analyzed using the “Epidemiological calculator” (R epicalc package). The Student t test was performed to calculate p value for continuous variables, and the Fisher exact test was used for comparative analysis of categorical variables. Hypothesis testing used the Student t test and Mann-Whitney U test, and local FDR (Efron et al. (2001) J. Am. Stat. Assoc. 96:1151-1160; herein incorporated by reference) to correct for multiple hypothesis testing issues. Clinical KD score was computed as previously described (Ling et al. (2013) The Journal of Pediatrics 162(1):183-188 e183; Ling et al. (2011) BMC Med. 9:130; herein incorporated by reference). The biomarker panel model was developed using the Random forest method with 5-fold cross validation. The predictive performance of each biomarker panel analysis was evaluated by ROC curve analysis (Zweig et al. (1993) Clinical Chemistry 39(4):561-577; Sing et al. (2005) Bioinformatics 21(20):3940-3941; herein incorporated by reference).

RESULTS

Study Design

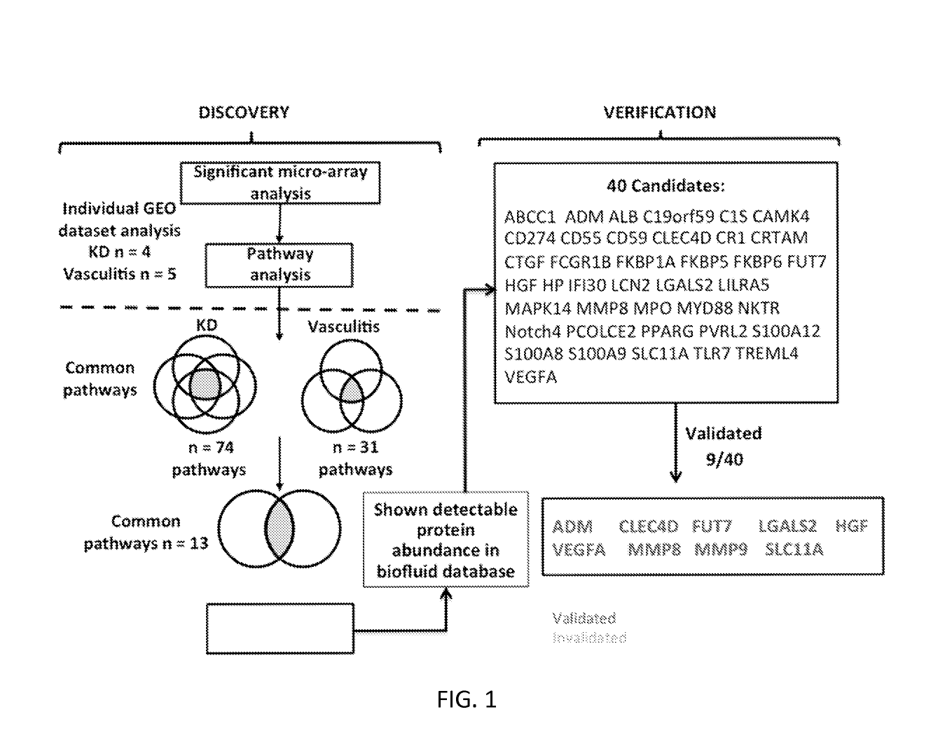

As shown in FIG. 1, our study was conducted in four phases: (1) The discovery phase. We performed gene-phenotype association analyses, calculating the counts of genes (n=37,314) to different KD heart lesion outcomes and vasculitis in PubMed (November 2012 release, >22 million articles) search fields; Venn diagram analysis was performed to identify the overlapping genes, which are the ones ranking top 0.5% largest counts in each of the gene-phenotype association analyses. In parallel, meta-analysis was performed on 7 GEO PBMC gene expression data sets to derive significant genes in both KD and other vasculitis data sets. 82 genes were found to be significant in both in silico analyses. (2) The verification phase. 40/82 genes were subjected to downstream verification analysis, using readily available commercial ELISA kits. (3) The KD diagnostic algorithm development phase and 5-fold cross validation testing phase. The panel classifier was tested with a cohort of 2 subjects (KD n=40, FC n=40) for its usefulness in KD diagnosis from FC subjects.

Novel KD Serum Biomarker Validation Using KD and FC Control Serum Samples

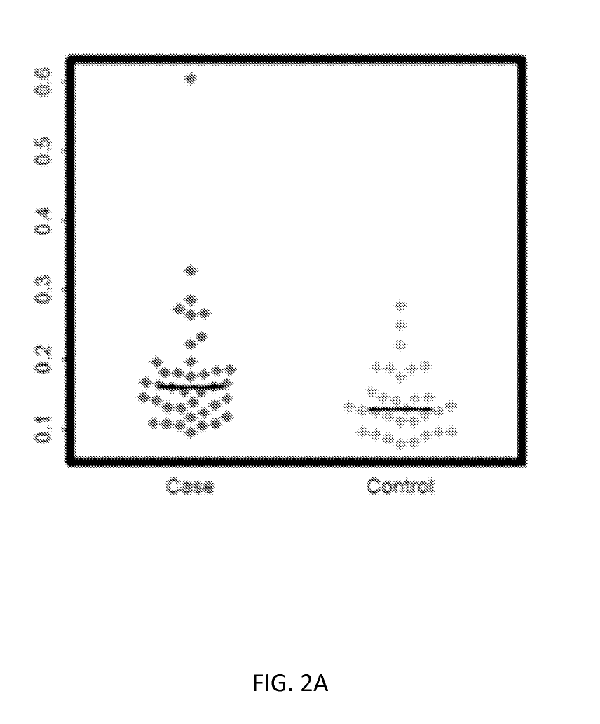

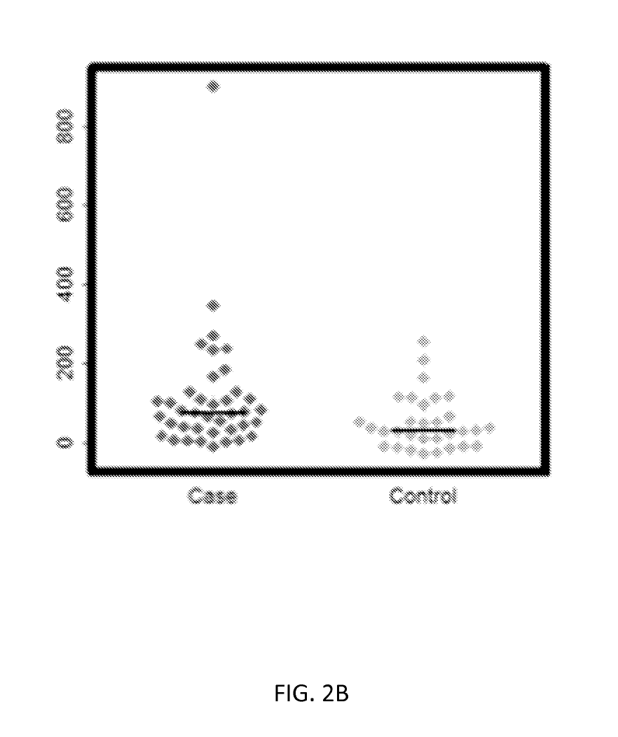

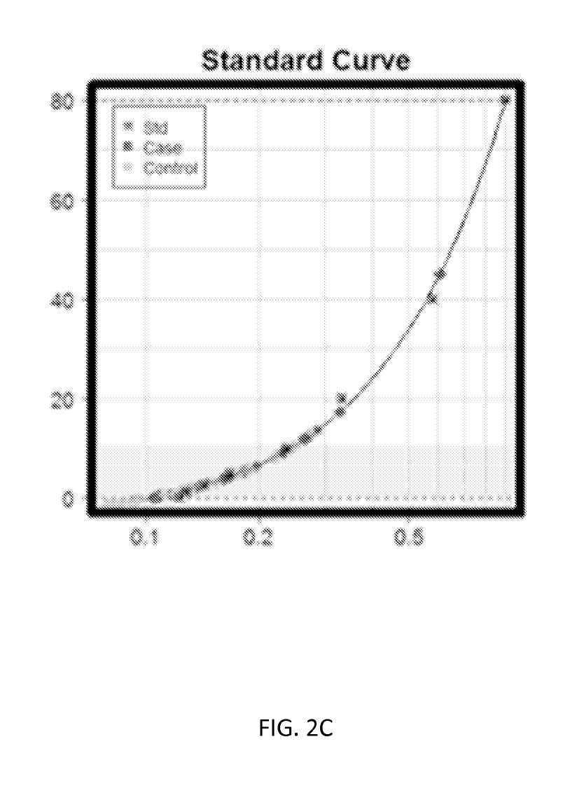

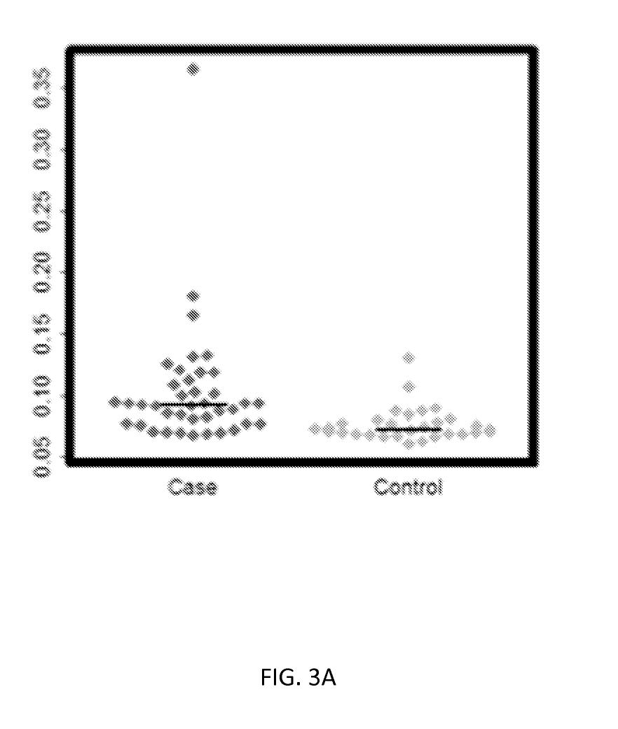

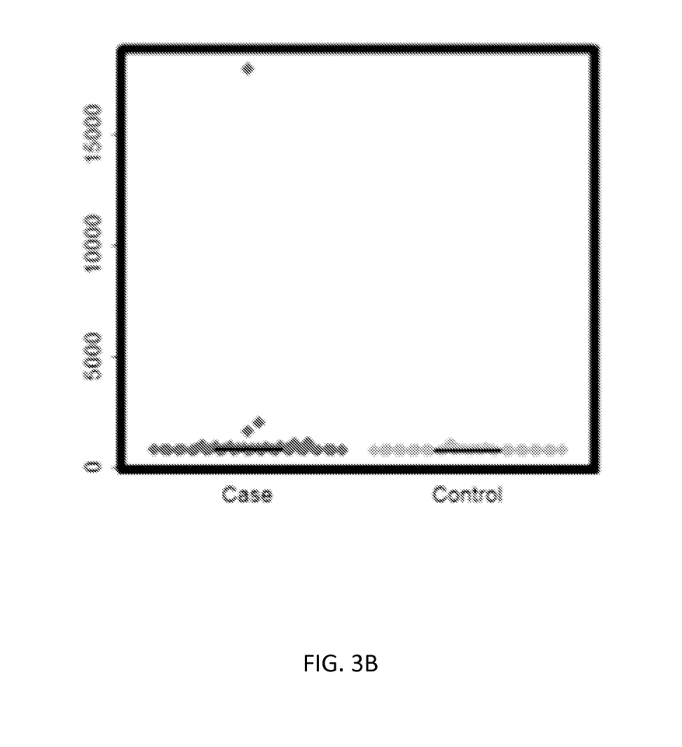

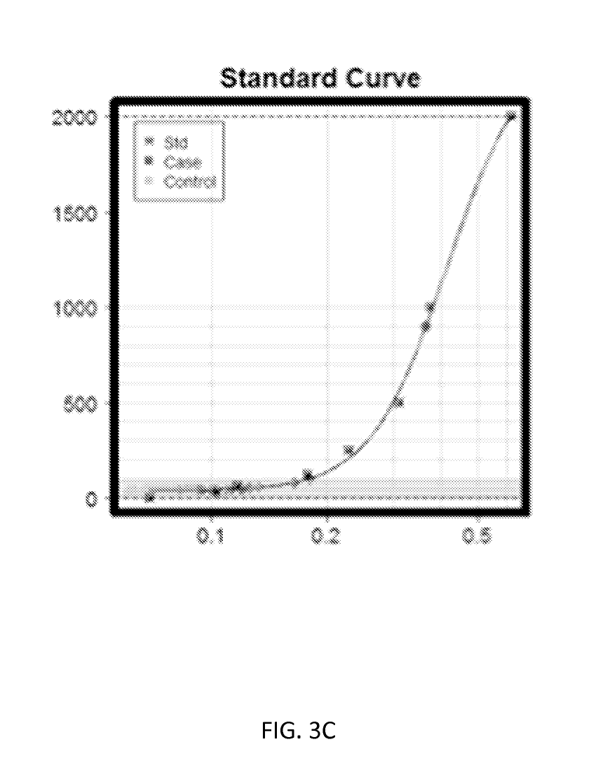

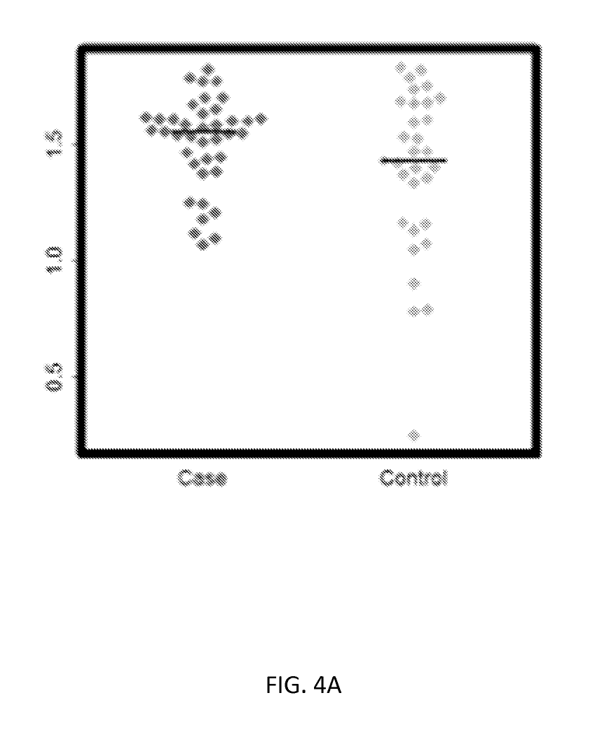

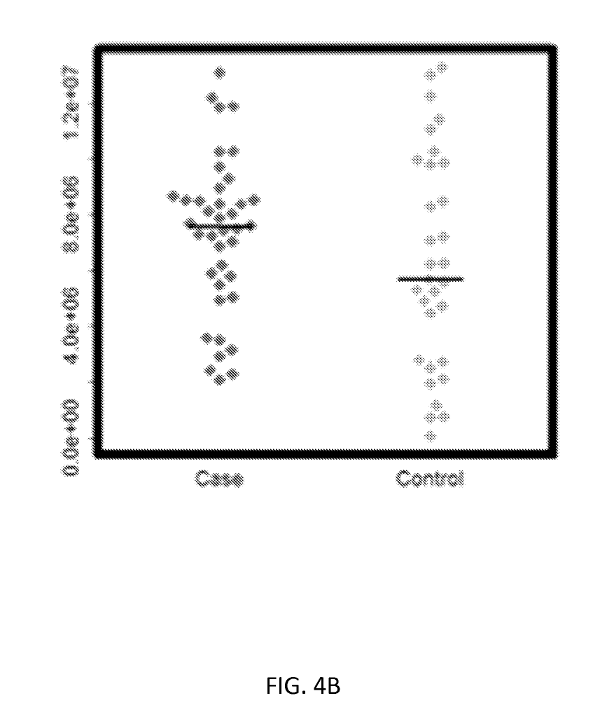

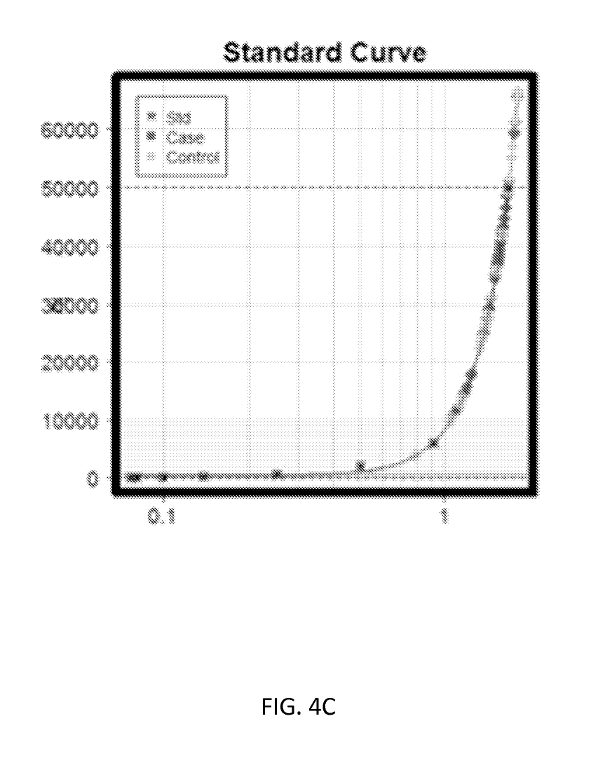

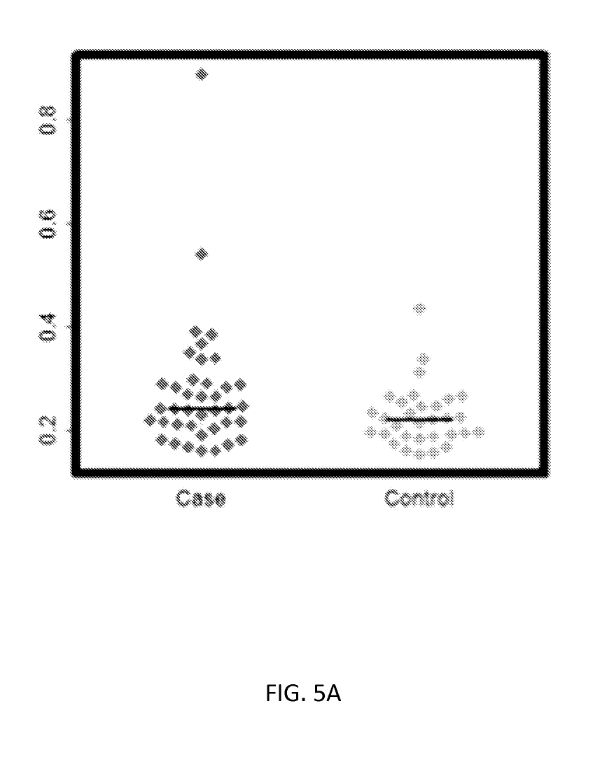

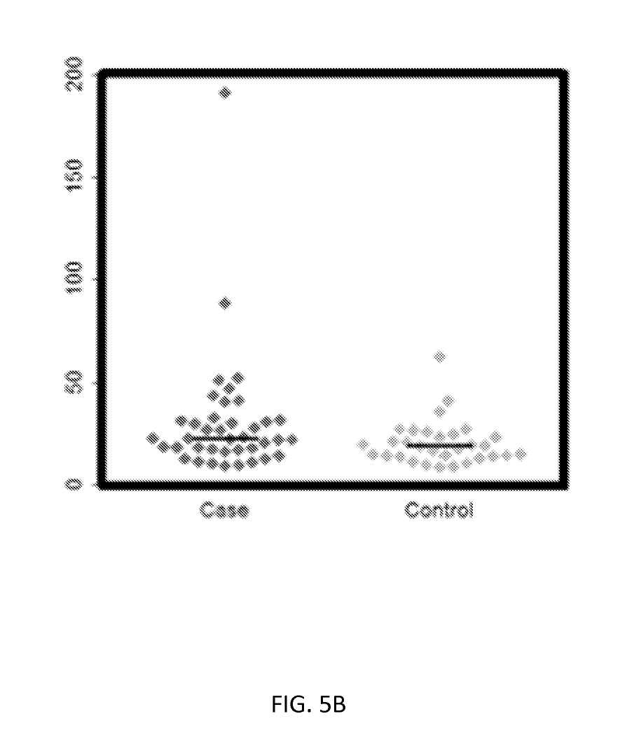

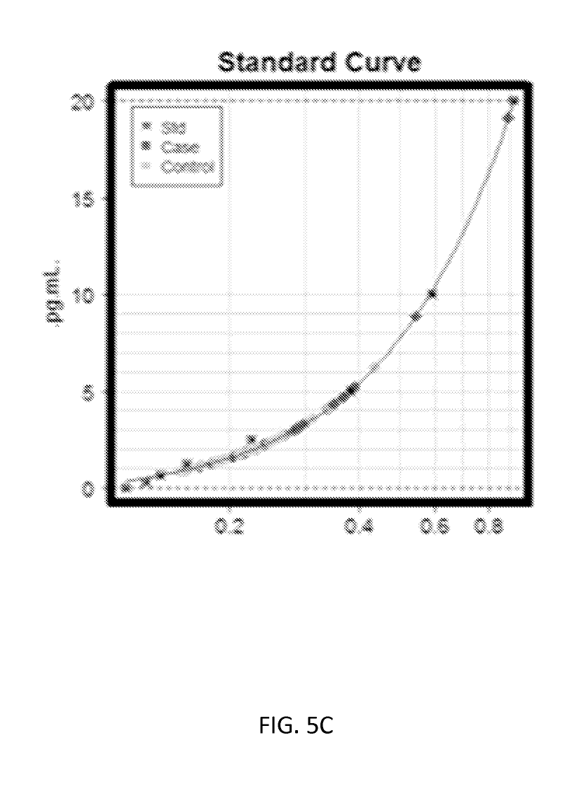

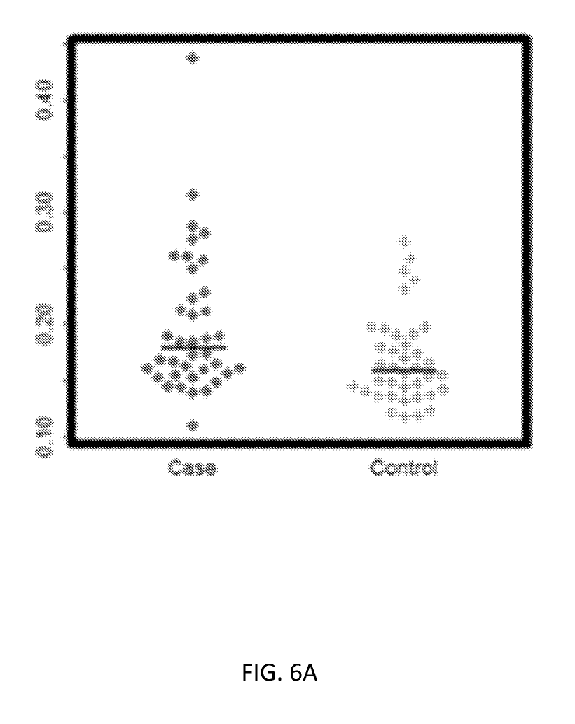

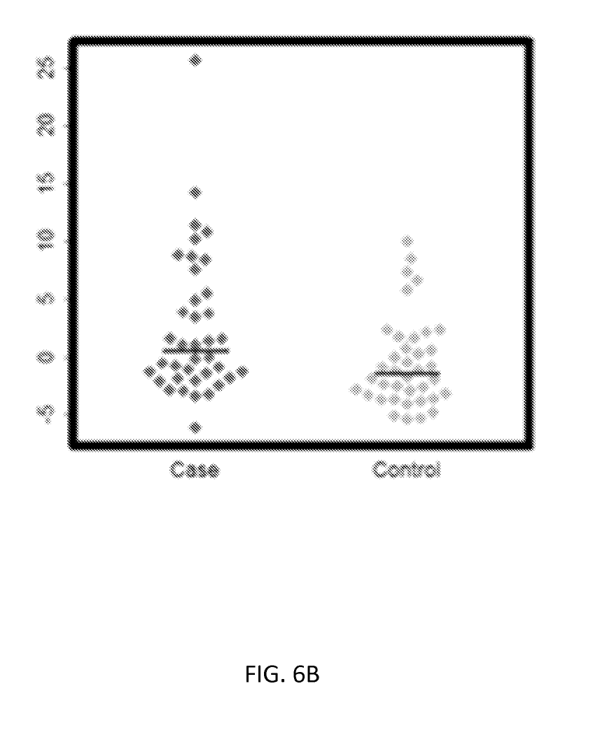

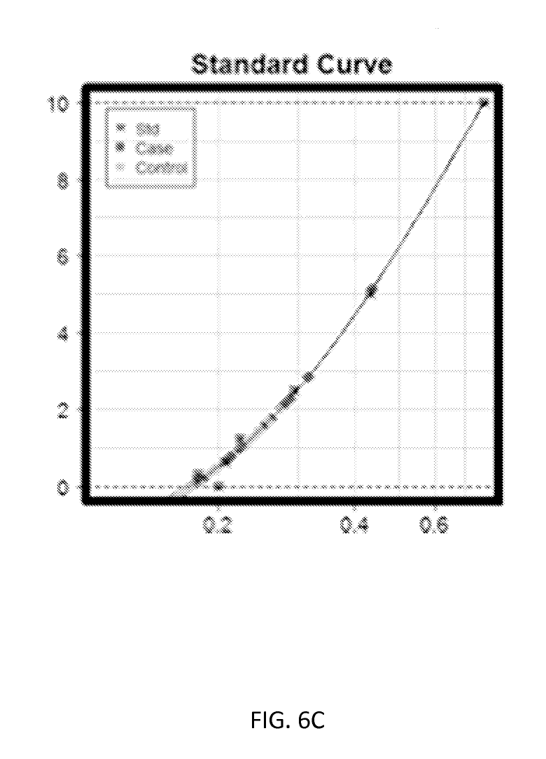

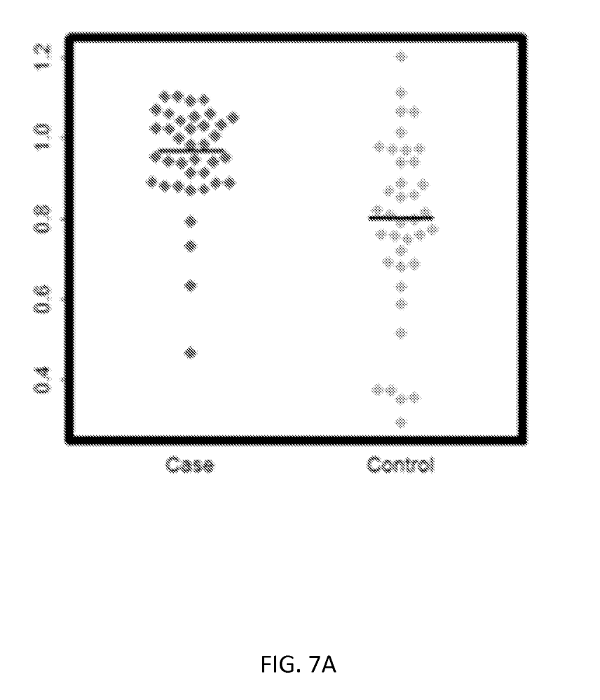

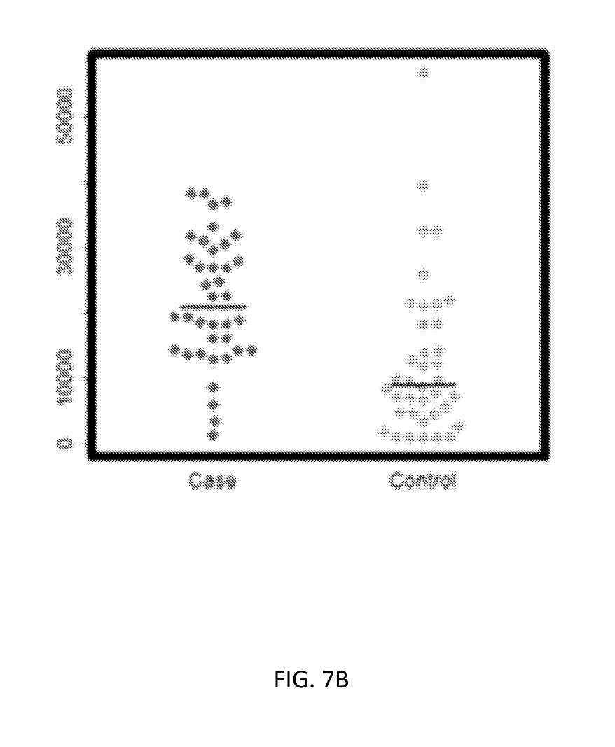

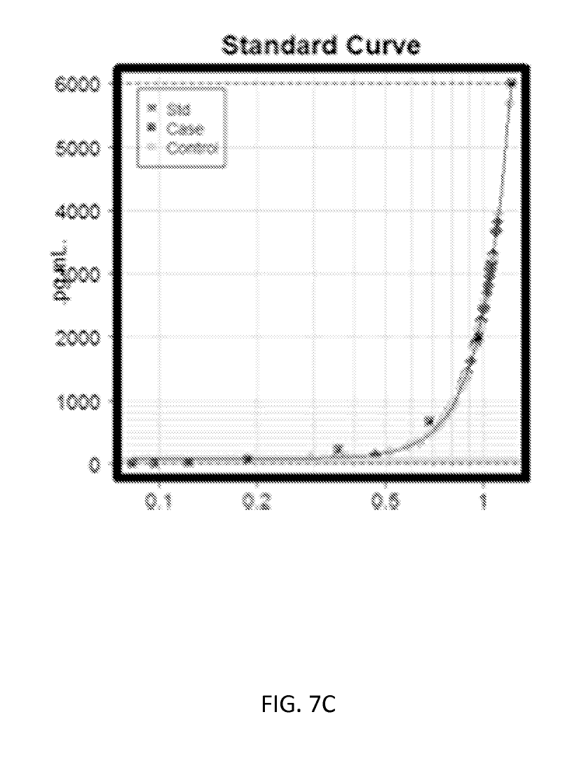

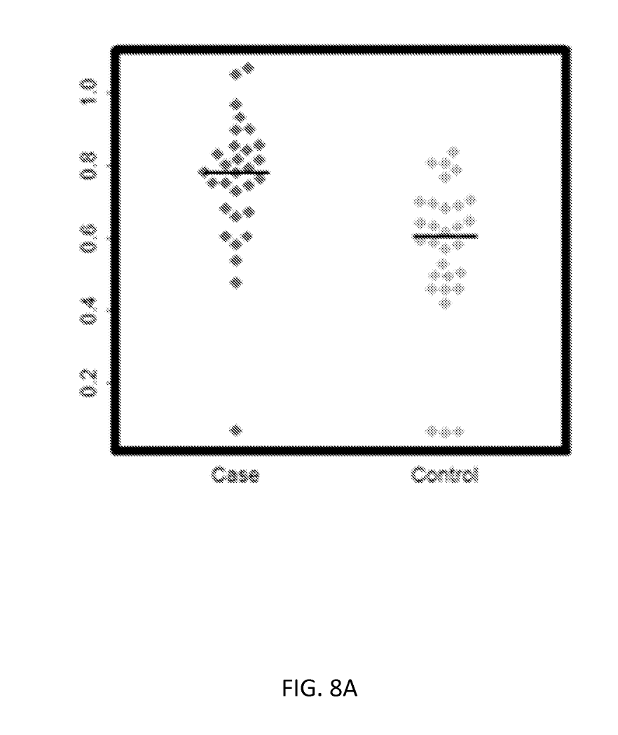

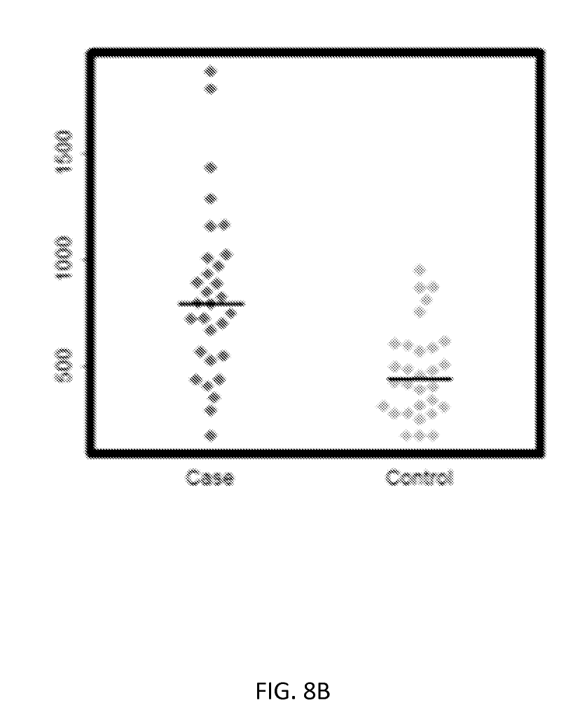

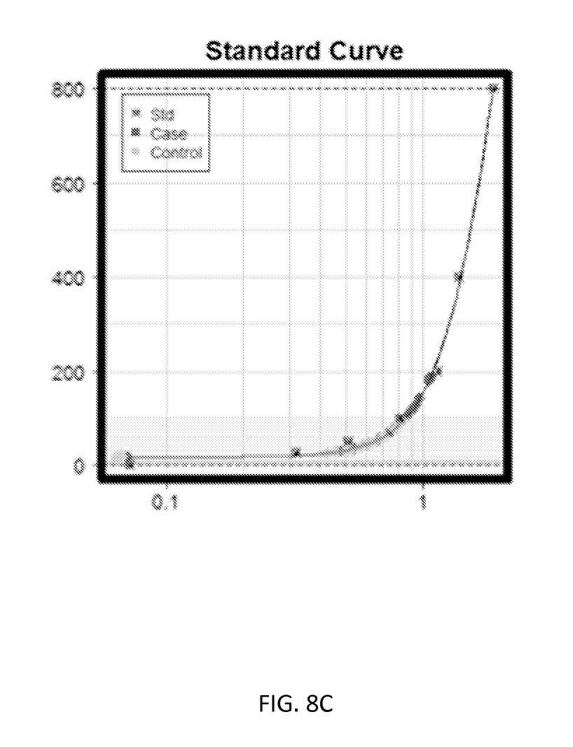

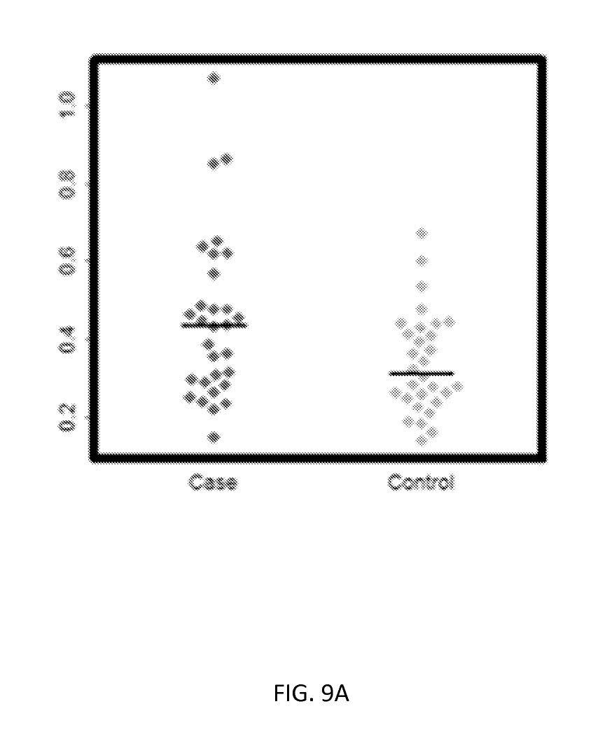

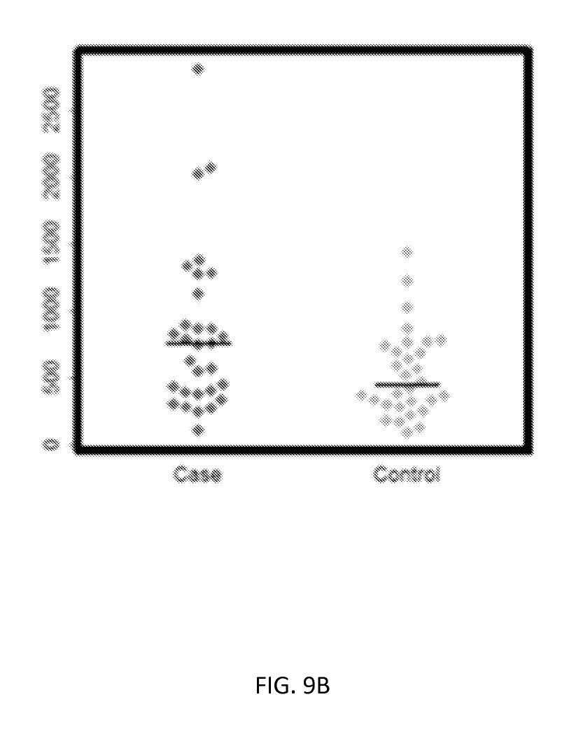

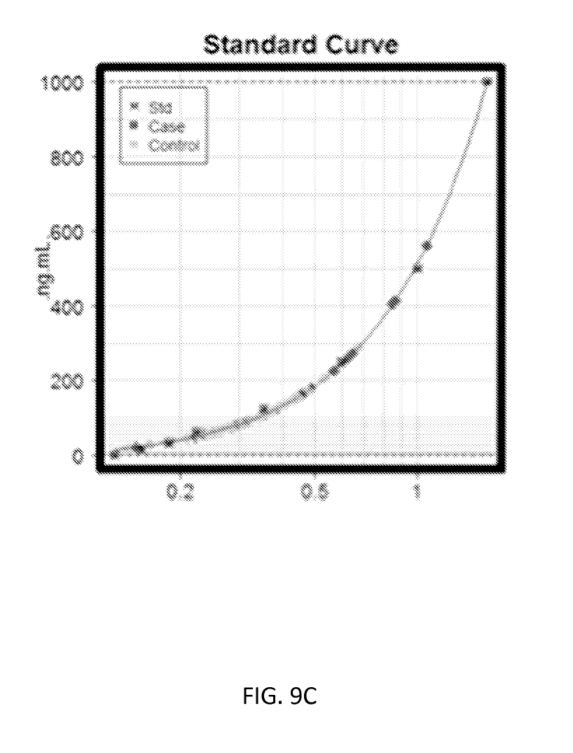

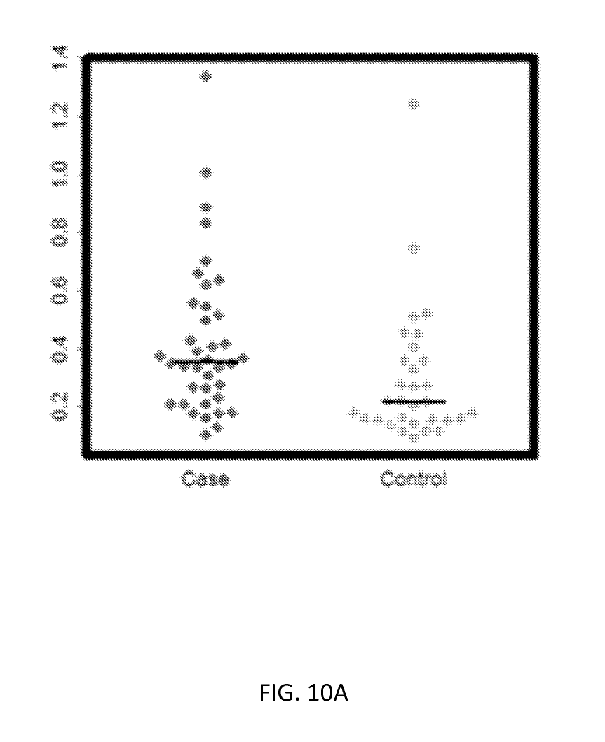

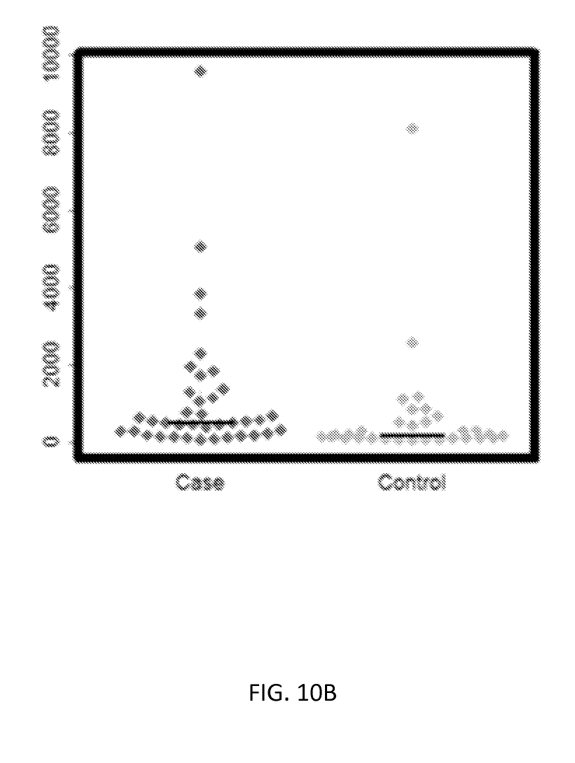

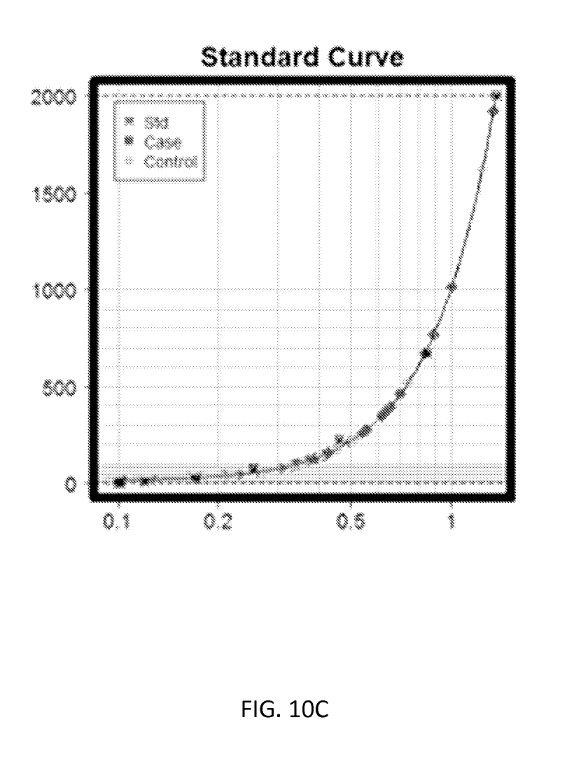

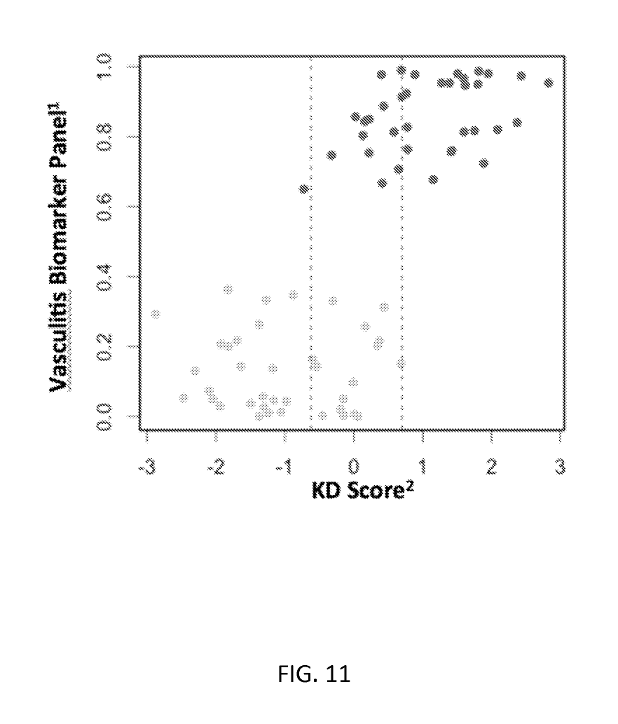

To identify whether the 40 KD biomarker candidates could enable development of an immediate practical diagnostic panel, based on available ELISA assays, biomarker candidates from in silico analyses were validated with available serum assays. Detailed with beeswarm plots and standard curves (FIGS. 2-10), a total of 9 proteins were validated by ELISA assays with Mann-Whitney test p values<0.05).

Our KD Serum Biomarker Panel (Analytes n=9) Classifier Effectively Diagnosing KD From FC Subjects

Using data from the 9-analyte ELISA assays, we constructed our KD diagnostic biomarker panel. We used the random forest method and a 5-fold cross validation method to develop the KD diagnostic classifier. FIG. 11 shows a plot of the new 9 analyte biomarker panel score as a function of the previous KD clinical scoring metric. This result indicates that the 9 analyte KD biomarker panel can be an effective diagnostic classifer to distinguish KD from FC subjects.

Discussion

In this study we sought a combination of 9 biomarkers and KD score analysis that could distinguish between KD subjects and FC subjects with sufficient accuracy to be clinically useful. Of the 9 biomarkers, SLC11A1 is a member of the solute carrier family 11 (proton-coupled divalent metal ion transporters) family and encodes a multi-pass membrane protein. The protein functions as a divalent transition metal (iron and manganese) transporter involved in iron metabolism and host resistance to certain pathogens. Mutations in this gene have been associated with susceptibility to infectious diseases such as tuberculosis and leprosy, and inflammatory diseases such as rheumatoid arthritis and Crohn's disease. Alternatively spliced variants that encode different protein isoforms have been described, but the full-length nature of only one has been determined.

In this first attempt at developing a vasculitis biomarker based panel for diagnosing KD, we focused on biomarkers that are included in commercial Elisa kits. Increased transcript abundance of matrix metalloproteinase (MMP) 9, a collagenase involved in the breakdown of extracellular matrix that may play a role in aneurysm formation, has been shown in children with acute KD compared to subjects with adenovirus infection and drug reactions (Popper et al. (2009) J. Infect. Dis. 200(4):657-666). Single nucleotide polymorphisms in vascular endothelial growth factor (VEGFA) have been associated with KD susceptibility, and serum levels are higher in acute compared to convalescent KD subjects and febrile controls (Breunis et al. (2012) Arthritis Rheum. 64(1):306-315; Maeno et al. (1998) Pediatric Research 44(4):596-599; Takeshita et al. (2005) Clin. Exp. Immunol. 139(3):575-579).

A central problem in the diagnosis of KD and the development of a diagnostic test is that the host response to inflammation involves pathways that are shared by many of the rash-fever illnesses that are in the differential diagnosis of KD, including adenovirus infection and scarlet fever (Barone (2000) Arch. Pediatr. Adolesc. Med. 154(5):453-456). Thus, proper controls from children with rash-fever illnesses that mimic KD are central to the development of a clinically useful diagnostic test. Previous studies have either used healthy children or children with febrile illnesses that do not mimic KD (such as pneumonia and bronchiolitis) as controls, thus discounting the importance of pre-test probability in the evaluation of a diagnostic test. As the pre-test probability of KD decreases in individuals being screened, the false positive rate will increase. The selection of febrile controls with diseases that mimic KD is critical, as it is this population in which the test will eventually be used in a clinical setting.

Future prospective trials of our novel KD diagnostic method and further characterization of the biological role of the 9 biomarkers in KD can lead to not only an effective KD diagnostic utility but also better understanding of KD pathophysiology.

### Example 2

### Determining Clinical Score for Diagnosis of Kawasaki Disease

Linear discriminant analysis (LDA) is used to stratify individual subjects based on a series of clinical exploratory variables. The R library MASS function ‘Ma’ (r-project.org/) is utilized. Coefficients of linear discriminants (LD1) are calculated as a measure of the association of each variable with the final diagnosis. The discriminant score is calculated from at least seven variables having the largest (absolute value) coefficients. Such clinical variables can include the number of days of fever at the time of a clinical visit (illDay), total white blood cell count (wbc), percentage of monocytes (monos), percentage of eosinophils (eos), percentage of eosinophils immature neutrophils (bands), concentration of hemoglobin (hgb), and concentration of C-reactive protein (crp). Patients are stratified into subgroups with low (≦5% likelihood KD), intermediate, and high (≧95% likelihood KD) clinical scores as described previously (Ling et al. (2011) BMC Med. 9:130; Ling et al. (2013) J. Pediatrics 162(1):183-188; herein incorporated by reference in their entireties). Patients with intermediate KD clinical scores can be further analyzed using biomarker expression profiles to improve diagnostic sensitivity and specificity.

While the preferred embodiments of the invention have been illustrated and described, it will be appreciated that various changes can be made therein without departing from the spirit and scope of the invention.

What is claimed is:

1. A biomarker panel for diagnosing KD comprising lectin galactoside-binding soluble 2 (LGALS2), fucosyltransferase 7 (FUT7), matrix metallopeptidase 9 (MMP9), adrenomedullin (ADM), C-type lectin domain family 4 member D (CLEC4D), matrix metallopeptidase 8 (MMP8), natural resistance-associated macrophage protein 1 (SLC11A1), vascular endothelial growth factor A (VEGFA), and hepatocyte growth factor (HGF).

2. The biomarker panel of claim 1, wherein the biomarker panel consists of LGALS2, FUT7, MMP9, ADM, CLEC4D, MMP8, SLC11A1, VEGFA, and HGF.

3. A method for diagnosing Kawasaki disease (KD) in a patient using the biomarker panel of claim 1, the method comprising:
a) obtaining a biological sample from the patient;
b) measuring levels of each biomarker of the biomarker panel of claim 1 in the biological sample; and
c) comparing the levels of each biomarker with respective reference value ranges for the biomarkers, wherein differential expression of the biomarkers of the biomarker panel of claim 1 in the biological sample compared to the reference value ranges for the biomarkers for a control subject indicate that the patient has a positive KD diagnosis.

4. The method of claim 3, further comprising:
a) determining a clinical score for the patient from measurements of at least seven clinical parameters for the patient, wherein the seven clinical parameters comprise duration of fever, concentration of hemoglobin in blood, concentration of C-reactive protein in blood, white blood cell count, percent eosinophils in blood, percent monocytes in blood, and percent immature neutrophils in blood; and
b) classifying the clinical score as a low risk KD clinical score, an intermediate risk KD clinical score, or a high risk KD clinical score, wherein a high risk KD clinical score or an intermediate risk KD clinical score in combination with a positive KD diagnosis based on the levels of the biomarkers indicate that the patient has KD.

5. The method of claim 3, further comprising distinguishing a diagnosis of KD from a diagnosis of febrile illness in the patient.

6. The method of claim 3, wherein the patient is a human being.

7. The method of claim 3, wherein measuring the biomarkers comprises performing an enzyme-linked immunosorbent assay (ELISA), a radioimmunoassay (RIA), an immunofluorescent assay (IFA), immunohistochemistry (IHC), a sandwich assay, magnetic capture, microsphere capture, a Western Blot, surface enhanced Raman spectroscopy (SERS), flow cytometry, or mass spectrometry.

8. The method of claim 7, wherein measuring a biomarker comprises contacting an antibody with the biomarker, wherein the antibody specifically binds to the biomarker, or a fragment thereof containing an antigenic determinant of the biomarker.

9. The method of claim 8, wherein the antibody is selected from the group consisting of a monoclonal antibody, a polyclonal antibody, a chimeric antibody, a recombinant fragment of an antibody, an Fab fragment, an Fab′ fragment, an F(ab′)₂ fragment, an Fᵥ fragment, and an scFᵥ fragment.

10. The method of claim 3, wherein the biological sample is serum, plasma, or blood.

11. A method of treating a patient suspected of having KD, the method comprising:
a) receiving a diagnosis of the patient according to the method of claim 3; and
b) administering a therapeutically effective amount of intravenous immunoglobulin (IVIG) to the patient if the patient has a positive KD diagnosis.

12. A method of treating a patient suspected of having KD, the method comprising:
a) receiving a diagnosis of the patient according to the method of claim 4; and
b) administering a therapeutically effective amount of intravenous immunoglobulin (IVIG) to the patient if the patient has a high risk KD clinical score or an intermediate risk KD clinical score and a positive KD diagnosis based on the levels of the biomarkers.

13. A method for evaluating the effect of an agent for treating KD in a patient using the biomarker panel of claim 1, the method comprising: measuring levels of each biomarker of the biomarker panel of claim 1 in samples derived from the patient before and after the patient is treated with said agent and comparing the levels of each biomarker with respective reference value ranges for each biomarker.

14. A method for monitoring the efficacy of a therapy for treating KD in a patient using the biomarker panel of claim 1, the method comprising: measuring levels of each biomarker of the biomarker panel of claim 1 in samples derived from the patient before and after the patient undergoes said therapy and comparing the levels of each biomarker with respective reference value ranges for each biomarker.

15. A kit for diagnosing KD comprising agents for measuring each biomarker of the biomarker panel of claim 1.

16. The kit of claim 15, further comprising information, in electronic or paper form, comprising instructions to correlate the levels of each of the biomarkers with KD.

17. The kit of claim 15, further comprising reagents for performing an immunoassay.

18. The kit of claim 17, wherein the agents comprise at least one antibody selected from the group consisting of an antibody that specifically binds to LGALS2, an antibody that specifically binds to FUT7, an antibody that specifically binds to MMP9, an antibody that specifically binds to ADM, an antibody that specifically binds to CLEC4D, an antibody that specifically binds to MMP8, an antibody that specifically binds to SLC11A1, an antibody that specifically binds to VEGFA, and an antibody that specifically binds to HGF.

19. A method for diagnosing Kawasaki disease (KD) in a patient, the method comprising:
a) obtaining a blood, plasma, or serum sample from the patient;
b) measuring levels of biomarkers comprising lectin galactoside-binding soluble 2 (LGALS2), fucosyltransferase 7 (FUT7), matrix metallopeptidase 9 (MMP9), adrenomedullin (ADM), C-type lectin domain family 4 member D (CLEC4D), matrix metallopeptidase 8 (MMP8), natural resistance-associated macrophage protein 1 (SLC11A1), vascular endothelial growth factor A (VEGFA), and hepatocyte growth factor (HGF) in the blood, plasma, or serum sample by performing an immunoassay; and
c) comparing the levels of each biomarker with reference values for each biomarker for a control subject, wherein differential expression of the biomarkers in the blood, plasma, or serum sample compared to the reference values indicate that the patient has a positive KD diagnosis.

20. The method of claim 19, wherein the immunoassay is an enzyme linked immunosorbent assay (ELISA).

21. The method of claim 19, wherein the method comprises contacting the blood, plasma, or serum sample with an antibody that specifically binds to LGALS2, an antibody that specifically binds to FUT7, an antibody that specifically binds to MMP9, an antibody that specifically binds to ADM, an antibody that specifically binds to CLEC4D, an antibody that specifically binds to MMP8, an antibody that specifically binds to SLC11A1, an antibody that specifically binds to VEGFA, and an antibody that specifically binds to HGF.

22. The method of claim 19, further comprising:
a) determining a clinical score for the patient from measurements of at least seven clinical parameters for the patient, wherein the seven clinical parameters comprise duration of fever, concentration of hemoglobin in blood, concentration of C-reactive protein in blood, white blood cell count, percent eosinophils in blood, percent monocytes in blood, and percent immature neutrophils in blood; and
b) classifying the clinical score as a low risk KD clinical score, an intermediate risk KD clinical score, or a high risk KD clinical score, wherein a high risk KD clinical score or an intermediate risk KD clinical score in combination with a positive KD diagnosis based on the levels of the biomarkers indicate that the patient has KD.
